(19) United States
(12) Patent Application Publication
(10) Pub. No.: US 2024/0316258 A1
(43) Pub. Date: Sep. 26, 2024

(54) Systems, Apparatus and Methods for Processing Autologous Blood for Reinfusion into a Patient

(71) Applicant: Retriever Medical, Inc., Las Vegas, NV (US)

(72) Inventors: Michael Bruce Horowitz, Naples, FL (US); Benjamin William Bobo, Las Vegas, NV (US); Brandon Mattew Repko, Mars, PA (US); Andreas Tzakis, Miami, FL (US)

(21) Appl. No.: 18/680,305

(22) Filed: May 31, 2024

Related U.S. Application Data
(63) Continuation-in-part of application No. 18/398,727, filed on Dec. 28, 2023.
Application No. 18/398,727 is a continuation-in-part of application No. 18/220,373, filed on Jul. 11, 2023.
(60) Provisional application No. 63/368,325, filed on Jul. 13, 2022.

(51) Int. Cl. A61M 1/36; B01D 29/56

(52) U.S. Cl. CPC A61M 1/3695; B01D 29/56; A61M 2202/0427; A61M 2202/0429; A61M 2202/0439; A61M 2202/0445; A61M 2206/14

(57) ABSTRACT

A blood reinfusion system for processing autologous blood for reinfusion into a patient. The system includes a blood reinfusion system having a blood processing canister with an internal fluid passageway. The internal fluid passageway includes a venturi region having a gravity-driven filtering system that is adapted to extract impurities from autologous blood without damaging the erythrocytes in the blood and, hence, inducing hemolysis of the erythrocytes in the blood, and loss of seminal native blood components, including native erythrocytes, platelets, white blood cells, plasma proteins and antibodies.

### CROSS-REFERENCES TO RELATED APPLICATIONS

The present application is a continuation-in-part of U.S. application Ser. No. 18/398,727, filed on Dec. 28, 2023, which is a continuation-in-part of U.S. application Ser. No. 18/220,373, filed on Jul. 11, 2023, which claims the benefit of U.S. Pat. App. No. 63/368,325, filed on Jul. 13, 2022.

### FIELD OF THE INVENTION

The present invention relates generally to systems and methods for processing blood. More particularly, the present invention relates to systems, apparatus and methods for processing autologous blood for reinfusion into a patient.

### BACKGROUND OF THE INVENTION

As is well established, blood loss is an inevitable aspect of many invasive surgical procedures and, if not managed or accounted for, can lead to various significant adverse physiological conditions.

Indeed, a loss of over 20% of blood volume (˜900 cc to 1000 cc) during a surgical procedure can cause hypovolemic shock and a loss of over 50% of blood volume (˜2250 cc to 3500 cc) can cause cardiac arrest.

Blood loss during a surgical procedure can also result in post-procedure anemia, which can, and often will, hinder recovery.

Various means have thus been employed to manage blood loss during a surgical procedure. The most common means is transfusion of blood during and after the procedure.

As is well established, there are, however, several significant drawbacks and disadvantages associated with transfusion of blood to a patient during and after a surgical procedure.

A major problem associated with a typical blood transfusion, is that such blood is typically non-autologous (i.e., donated by another person), and thus, can induce various adverse physiological events, such as antigen reactions and disease transfer, if not properly screened.

Various autologous blood processing and recycling systems have thus been developed to address blood loss during a surgical procedure. Such systems include the Cell Saver® Elite®+ autotransfusion system developed by Haemonetics, the CATSmart® continuous autotransfusion system developed by Fresenius Kabi, the XTRA® autotransfusion system developed by LivaNova, and the autoLog® autotransfusion system developed by Medtronic.

The noted systems typically include means for collecting autologous blood from a patient during a surgical procedure, means for processing the collected autologous blood, i.e., extracting impurities from the collected autologous blood and/or adding pharmacological agents and/or biologics to the collected autologous blood, and means for reinfusing the processed autologous blood into the patient.

As discussed below, there are similarly numerous drawbacks and disadvantages associated with the noted “blood reinfusion” systems.

A major disadvantage associated with the noted blood reinfusion systems is that the blood processing means of the systems; particularly, the means for extracting impurities from the blood, can, and often will, damage the erythrocytes (i.e., red blood cells) in the collected “autologous” blood, which can, and often will, compromise the quality of the blood.

A further major disadvantage associated with the noted blood reinfusion systems is that the blood processing means typically includes mixing the collected “autologous” blood with a physiological solution (e.g., saline or Ringer's Solution) and centrifuging the mixed blood to isolate and recover the erythrocytes for reinfusion into the patient. The lighter portion of centrifuged mixed blood (i.e., the lighter plasma and buffy coat of the whole blood), which contains platelets, white blood cells, plasma proteins, and antibodies, are typically discarded as waste. The processed and, hence, reinfused blood is thus devoid of the highly important platelets, white blood cells, plasma proteins and antibodies.

A further disadvantage associated with the noted blood reinfusion systems is that such systems typically comprise large, complex equipment that is very difficult to operate and require multiple specialized technicians to operate. The systems thus often require advanced planning prior to use, including scheduling specialized technicians trained to set up and use the systems, and, hence, are also suboptimal for emergency use, e.g., instances of unexpected blood loss during a medical procedure or military combat.

A further disadvantage associated with the noted blood reinfusion systems is that they are typically not configured and/or adapted for use in sterile environments.

A further disadvantage associated with the noted blood reinfusion systems is the high costs associated with reinfusing blood into a patient therewith, i.e., reinfusion system acquisition and labor costs. As a result, such systems are typically not economically feasible for use during surgical procedures in developing countries.

It would thus be desirable to provide improved blood reinfusion systems that substantially reduce or eliminate the drawbacks and disadvantages associated with conventional blood reinfusion systems.

It is therefore an object of the present invention to provide improved blood reinfusion systems that substantially reduce or eliminate the drawbacks and disadvantages associated with conventional blood reinfusion systems.

It is another object of the present invention to provide improved blood reinfusion systems adapted to process autologous blood for reinfusion into a patient with minimal, if any, effect on the quality of the blood.

It is another object of the present invention to provide blood processing systems adapted to process non-autologous blood for transfusion into a patient with minimal, if any, effect on the quality of the blood.

It is another object of the present invention to provide improved blood reinfusion systems adapted to process autologous blood for reinfusion into a patient without damaging the erythrocytes in the blood.

It is another object of the present invention to provide improved blood reinfusion systems adapted to process autologous blood with minimal blood component loss; specifically, platelet, white blood cell, plasma protein, and antibody loss.

It is another object of the present invention to provide improved blood reinfusion systems configured and adapted for use in sterile environments.

It is another object of the present invention to provide improved blood reinfusion systems that are simple to use and can be easily operated manually by a single operator.

It is another object of the present invention to provide improved blood reinfusion systems that can be promptly employed in emergency situations.

It is another object of the present invention to provide improved blood reinfusion systems that can be readily employed in a multitude of surgical and interventional medical procedures.

### SUMMARY OF THE INVENTION

The present invention is generally directed to systems, apparatus and methods for processing blood, more particularly, autologous blood for reinfusion into a patient.

In some embodiments of the invention, there are thus provided systems for processing autologous blood for reinfusion into a patient (referred to hereinafter as “blood reinfusion systems”).

In some embodiments of the invention, the blood reinfusion systems comprise a blood processing canister comprising a housing having an internal fluid passageway and a bottom blood collection chamber,
the internal fluid passageway sized and configured to receive blood therein and allow the blood to be transmitted therethrough, the internal fluid passageway in communication with the bottom blood collection chamber,
the internal fluid passageway comprising a venturi region, the venturi region comprising an inlet region, a restricted flow path region and an outlet region,
the venturi region further comprising a gravity-driven blood filtering system adapted to extract impurities from the blood, whereby purified whole blood is obtained,
the internal fluid passageway configured and adapted to transmit the purified whole blood to the bottom blood collection chamber.

In a preferred embodiment, the venturi inlet region comprises an inlet cone angle in the range of approximately 36.0° to approximately 56.0°.

In a preferred embodiment, the venturi outlet region comprises an outlet cone angle in the range of approximately 10.0° to approximately 45.0°.

In a preferred embodiment, blood flow through the internal fluid passageway and hence, venturi region exhibits a substantially laminar flow pattern, whereby the risk of shear forces imparted to blood and, hence, induced hemolysis of the erythrocytes in the blood is minimized.

In a preferred embodiment, the purified whole blood comprises a retained content of native blood components, including native erythrocytes, platelets, white blood cells, plasma proteins and antibodies, of at least 98.0% of original native blood component content.

In a preferred embodiment, the purified whole blood also comprises an impurity content of less than 0.05%.

In a preferred embodiment, the gravity-driven blood filtering system comprises a plurality of gravity-driven filters.

In a preferred embodiment, the plurality of gravity-driven filters comprises first, second, third and fourth filters.

In a preferred embodiment, the first filter comprises a pore size less than 10.0 mm, the second filter comprises a pore size less than 5.0 mm, the third filter comprises a pore size less than 300.0 micron, and the fourth filter comprises a pore size less than 100.0 micron.

In a preferred embodiment, the gravity-driven filters comprise a conical shape comprising a cone angle in the range of approximately 25.0° to approximately 35.0° relative to a horizontal plane.

In a preferred embodiment, blood flow through the internal fluid passageway comprises a flow rate of at least 500.0 cc/min.

In some embodiments, the blood processing canister further comprises sensor means adapted to monitor flow of the processed blood through the blood filtering system.

In some embodiments of the invention, there are also provided methods for processing autologous blood for reinfusion into a patient.

In one embodiment of the invention, the method for processing autologous blood for reinfusion into a patient comprises the steps of:
providing the blood reinfusion system described above,
providing means for extracting autologous blood from the patient and delivering the autologous blood to the blood reinfusion system,
processing the autologous blood with the blood reinfusion system by transmitting the blood into and through the venturi region and, thereby, gravity-driven blood filtering system of the blood reinfusion system, whereby purified, whole autologous blood is obtained.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 depicts a schematic illustration of one embodiment of a blood reinfusion system, in accordance with the invention;

FIG. 2 depicts a front plan view of the blood reinfusion system depicted in FIG. 1, in accordance with the invention;

FIG. 3A depicts a front plan view of one embodiment of a suction container, in accordance with the invention;

FIG. 3B depicts a top plan view of one embodiment of a suction container filter, in accordance with the invention;

FIG. 4 depicts a front plan view of one embodiment of a blood filter assembly, in accordance with the invention;

FIG. 5 depicts a schematic illustration of another embodiment of a blood reinfusion system, in accordance with the invention;

FIG. 6 depicts a schematic illustration of another embodiment of a blood reinfusion system, in accordance with the invention;

FIG. 7 depicts a front plan view of one embodiment of a blood collection container, in accordance with the invention;

FIG. 8 depicts a schematic illustration of another embodiment of a blood reinfusion system, in accordance with the invention;

FIG. 9 depicts a schematic illustration of another embodiment of a blood reinfusion system, in accordance with the invention;

FIG. 10 depicts a schematic illustration of another embodiment of a blood reinfusion system, in accordance with the invention;

FIG. 11 depicts a front plan view of the blood reinfusion system depicted in FIG. 10, in accordance with the invention;

FIG. 12 depicts a front plan view of one embodiment of a blood collection or transfer bag, in accordance with the invention;

FIG. 13 depicts a schematic illustration of another embodiment of a blood reinfusion system, in accordance with the invention;

FIG. 14 depicts a schematic illustration of another embodiment of a blood reinfusion system, in accordance with the invention;

FIG. 15 depicts a schematic illustration of another embodiment of a blood reinfusion system, in accordance with the invention;

FIGS. 16A and 16C depict sectional front plan views of another embodiment of a blood reinfusion system, in accordance with the invention;

FIG. 16B depicts a top plan view of one embodiment of the system top depicted in FIGS. 16A and 16C, in accordance with the invention;

FIGS. 16D and 16E depict front plan views of the venturi region of the blood reinfusion system depicted in FIGS. 16A and 16C;

FIG. 17 depicts a perspective view of one embodiment of a thrombectomy system, in accordance with the invention;

FIG. 18A depicts an illustration of thrombosed bovine blood, in accordance with the invention;

FIG. 18B depicts an illustration of blood impurities captured in a filter of a blood filter assembly, in accordance with the invention; and

FIG. 18C depicts an illustration of a 40 micron filter after a portion of blood processed in the blood filter assembly depicted in FIG. 4 is filtered therewith, in accordance with the invention.

### DETAILED DESCRIPTION OF THE PREFERRED EMBODIMENT

Before describing the present invention in detail, it is to be understood that this invention is not limited to particularly exemplified systems, apparatus, structures or methods as such may, of course, vary. Thus, although a number of systems, apparatus, structures and methods similar or equivalent to those described herein can be used in the practice of the present invention, the preferred systems, apparatus, structures and methods are described herein.

It is to be understood that the terminology used herein is for the purpose of describing particular embodiments of the invention only and is not intended to be limiting. The present invention is thus to be accorded the widest scope encompassing numerous alternatives, modifications and equivalents consistent with the principles and features disclosed.

It is also to be understood that language used in this specification should not be interpreted as a general disavowal of any one specific embodiment or used to limit the claims beyond the meaning of the terms used therein.

Further, unless defined otherwise, all technical and scientific terms used in this specification have the same meaning as commonly understood by one having ordinary skill in the art to which the invention pertains.

It is also understood that the general principles defined herein can be applied to other embodiments and applications without departing from the spirit and scope of the invention.

It is also understood that any feature or component described in association with a specific embodiment may be used and implemented with any other embodiment unless clearly indicated otherwise.

Further, all publications, patents and patent applications cited herein, whether supra or infra, are hereby incorporated by reference in their entirety.

### Definitions

The term “surgical procedure”, as used herein, means an invasive medical procedure characterized by purposeful/deliberate access to the body via an incision or percutaneous puncture, where blood can, and often will be exhibited.

The term “surgical procedure”, as used herein, thus includes, without limitation, the following surgical procedures: cardiac surgery procedures, such as coronary artery bypass grafting (CABG), valve replacement and repair, and aortic aneurysm repair; orthopedic surgery procedures; spinal surgery procedures; neurosurgery procedures, such as craniotomy procedures; tumor resection procedures; organ transplant procedures; and trauma surgery procedures, such as trauma resuscitation and emergency surgical hemostasis.

The term “surgical procedure”, as used herein, also includes, without limitation, interventional cardiology procedures, such as coronary angiography, percutaneous coronary intervention (PCI), angioplasty, coronary stent placement, atherectomy, and transcatheter aortic valve replacement (TAVR); interventional vascular surgery procedures, such as endovascular aneurysm repair; interventional neurosurgery procedures, such as aneurysm coiling and arteriovenous malformation (AVM) procedures; and interventional trauma procedures.

The term “impurity”, as used herein in connection with blood, means and includes, without limitation, blood clots, tissue debris, hair, foreign particles, activated coagulation factors, denatured proteins, plasma free hemoglobin, and any other fluid (e.g., irrigation fluid) introduced into the surgical site by medical personnel.

The terms “thrombus” and “occlusion” are used interchangeably herein and mean and include unwanted or undesired material disposed in a patient's veins or arteries that is partially or completely obstructing the flow of blood.

The term “whole blood”, as used herein, means blood that comprises all seminal native blood components, including erythrocytes (red blood cells), native platelets, white blood cells, plasma proteins and antibodies.

The term “purified blood”, as used herein, means blood substantially devoid of impurities and unwanted cellular and blood components.

The term “gravity-driven”, as used herein in connection with filter systems and filters of the invention, means and includes filtering of blood therethrough solely by the force of gravity and, hence, without any induced positive or negative forces.

The terms “one embodiment”, “one aspect”, “an embodiment” and “an aspect”, as used herein, mean that a particular feature, structure, or characteristic described in connection with the embodiment can be included in at least one embodiment and not that any particular embodiment is required to have a particular feature, structure or characteristic described herein unless set forth in the claim.

The phrase “in one embodiment” or similar phrases employed herein do not limit the inclusion of a particular element of the invention to a single embodiment. The element can thus be included in other, or all embodiments discussed herein.

The term “substantially”, as used herein, means and includes the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing nearly all the length of a lumen would be substantially enclosed, even if the distal end of the structure enclosing the lumen had a slit or channel formed along a portion thereof.

Use of the term “substantially” is equally applicable when used in a negative connotation to refer to the complete or near complete lack of an action, characteristic, property, state, structure, item, or result. For example, a structure which is “substantially free of” a bottom would either completely lack a bottom or so nearly completely lack a bottom that the effect would be effectively the same as if it completely lacked a bottom.

The term “comprise” and variations of the term, such as “comprising” and “comprises,” means “including, but not limited to” and is not intended to exclude, for example, other components, elements or steps.

The following disclosure is provided to further explain in an enabling fashion the best modes of performing one or more embodiments of the present invention. The disclosure is further offered to enhance the understanding and appreciation for the inventive principles and advantages thereof, rather than to limit in any manner the invention. The invention is defined solely by the appended claims, including any amendments made during the pendency of this application, and all equivalents of those claims as issued.

As indicated above, described herein are blood reinfusion systems, apparatus and methods for processing autologous blood for reinfusion into a patient.

It is, however, to be understood that, although the systems, apparatus and methods are primarily described in connection with processing autologous blood for reinfusion into a patient, the systems, apparatus and methods are not limited to such application. According to the invention, the systems, apparatus and methods of the invention can also be readily employed to process non-autologous blood for transfusion into a patient.

As discussed in detail herein, the blood reinfusion systems, apparatus and methods of the invention provide numerous significant advantages over conventional blood reinfusion systems. Among the advantages are the following:
means for processing autologous blood with minimal, if any, effect on the quality of the blood;
means for processing autologous blood without damaging the erythrocytes in the blood;
means for processing autologous blood with minimal blood component loss; specifically, erythrocyte, platelet, white blood cell, plasma protein, and antibody loss;
blood reinfusion systems, apparatus and methods that can be employed in sterile environments; and
blood reinfusion systems, apparatus and methods that are simple to use and can be easily operated manually by a single operator.

A further advantage of the blood reinfusion systems, apparatus and methods of the invention is that they can be promptly and readily employed during a multitude of surgical and interventional medical procedures, including, without limitation, invasive cardiac procedures, such as coronary artery bypass grafting (CABG), valve replacement and repair, and aortic aneurysm repair; orthopedic surgery procedures; spinal surgery procedures; neurosurgery procedures, such as craniotomy procedures; tumor resection procedures; organ transplant procedures; thrombectomy procedures; interventional cardiology procedures, such as percutaneous coronary intervention (PCI) and transcatheter aortic valve replacement (TAVR); interventional vascular procedures, such as endovascular aneurysm repair; interventional neurosurgery procedures, such as aneurysm coiling and arteriovenous malformation (AVM) procedures; and various trauma procedures.

The blood reinfusion systems, apparatus and methods of the invention can also be readily employed at temporary trauma sites, such as a field hospital or trauma center in a combat zone, and permanent trauma treatment facilities and centers, such as in an emergency room or an intensive care unit (ICU).

As discussed in detail herein, in some embodiments, the blood reinfusion systems of the invention comprise (i) first blood collection means adapted to receive aspirated autologous blood from a patient, (ii) blood processing means in communication with the blood collection means adapted to process, i.e., remove impurities from, the autologous blood, and (iii) second blood collection means in communication with the processing means adapted to receive the autologous blood after processing.

As also discussed in detail herein, in some embodiments, the blood reinfusion systems of the invention comprise multiple separate first blood collection means and/or multiple separate second blood collection means.

In some embodiments, the blood reinfusion systems of the invention comprise modular systems, e.g., the first blood processing means and/or second blood processing means detachably coupled to the blood processing means.

As discussed in detail below, in some embodiments, the blood reinfusion systems of the invention solely comprise blood processing means adapted to process autologous and non-autologous blood for infusion into a patient.

In some embodiments, the blood reinfusion systems of the invention further comprise sensor means adapted to monitor the volume of aspirated autologous blood in the first blood collection means.

In some embodiments, the blood reinfusion systems of the invention further comprise sensor means adapted to monitor blood flow through the blood processing means.

In some embodiments, the blood reinfusion systems of the invention further comprise (i) aspiration means configured and adapted to collect autologous blood from a surgical site of a patient and (ii) control means programmed to control the aspiration means.

In some embodiments of the invention, the blood reinfusion systems further comprise integral transfusion means for reinfusing the processed (i.e., purified) autologous blood into the patient.

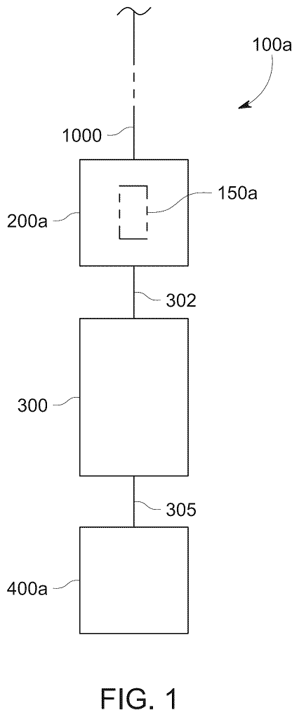

Referring now to FIG. 1, there is depicted a schematic illustration of one embodiment of a blood reinfusion system of the invention (denoted “100a”).

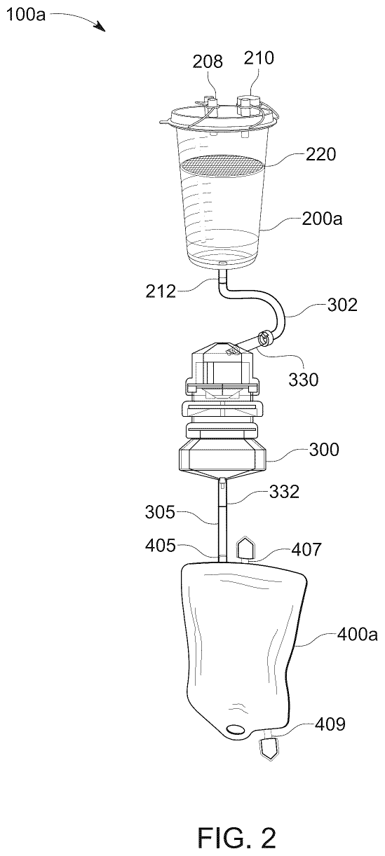

As depicted in FIGS. 1 and 2 and indicated above, in the illustrated embodiment, the blood reinfusion system 100a comprises first blood collection means (denoted “200a” and referred to herein as a “suction canister”), blood processing means (denoted “300” and referred to herein as a “primary blood filter system”), and second blood collection means (denoted “400a” and referred to herein as a “blood collection container”)

Each of the noted system components is described in detail below.

### Suction Canister

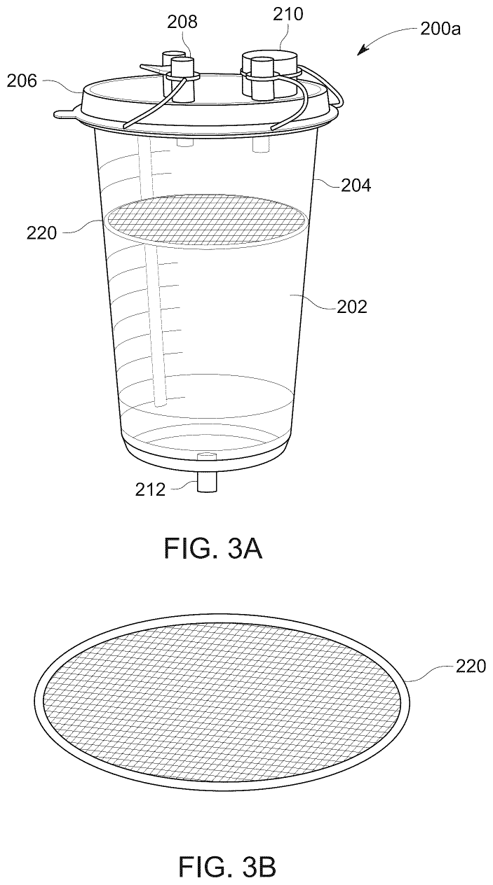

Referring now to FIGS. 2, 3A and 3B, suction canister 200a will be described in detail.

As depicted in FIG. 3A, in a preferred embodiment, suction canister 200a comprises a canister housing 220 and a top cap 206, which, according to the invention and depicted in FIG. 3A, is sized and configured to sealably engage the top open portion 204 of the canister housing 220.

As further depicted in FIG. 3A, the suction canister cap 206 comprises a blood inlet 208, which is adapted to communicate with and, hence, receive autologous blood from external aspiration means 1000, such as an aspiration catheter.

As additionally depicted in FIG. 3A, the suction canister 200a further comprises an internal fluid reservoir 202 and a blood outlet 212 in communication therewith, which, as discussed below, is sized and configured to receive the inlet line (i.e., conduit means) 302 of the primary blood filter system 300 to facilitate communication of the suction canister 200a and, hence, autologous blood contained in the suction canister reservoir 202, with the filter system 300.

In a preferred embodiment, the suction canister 200a (and suction canister 200b, referenced herein) further comprises blood pre-processing means adapted to pre-process autologous blood received therein, i.e., autologous blood aspirated from a patient.

In some embodiments, the canister pre-processing means comprises an internal filter adapted to extract impurities from autologous blood received therein, such as canister internal filter 220 depicted in FIGS. 3A and 3B.

According to the invention, the internal filter 220 can comprise any pore size. In a preferred embodiment, the internal filter 220 comprises a pore size less than approximately 10.0 mm.

In a preferred embodiment, the internal filter 220 comprises a domed (or convex) or cone shape and is positioned in the suction canister 200a in an upward trajectory.

According to the invention, the canister pre-processing means and, hence, suction catheter 200a (and suction canister 200b) can also comprise a plurality of filters.

According to the invention, the filters can comprise any suitable pore size, including, without limitation, the filter pore sizes referenced above.

Thus, in some embodiments of the invention, the suction canister pre-processing means comprises three (3) separate filters: a first filter comprising a pore size less than approximately 5.0 mm, a second filter comprising a pore size less than approximately 300.0 micron, and a third filter comprising a pore size less than approximately 50.0 micron.

In some embodiments of the invention, the suction canister pre-processing means comprises four (4) separate filters: a first filter comprising a pore size less than approximately 5.0 mm, a second filter comprising a pore size less than approximately 3.0 mm, a third filter comprising a pore size less than approximately 300.0 micron, and a fourth filter comprising a pore size less than approximately 50.0 micron.

As depicted in FIG. 1, in some embodiments of the invention, the suction canister 200a further comprises first sensor means 150a adapted to monitor autologous blood volume in the suction canister reservoir 202.

According to the invention, the suction canisters of the invention, i.e., suction canister 200a (and suction canister 200b) can comprise any configuration and size.

In a preferred embodiment, the suction canister 200a is sized and configured to receive and contain in the range of 200.0 ml to 1000.0 ml of fluid, e.g., autologous blood. In some embodiments of the invention, the suction canister 200a is sized and configured to receive and contain approximately 500.0 ml of fluid.

As indicated above and discussed in detail below, in some embodiments of the invention, the blood reinfusion system 100a comprises two suction canisters (e.g., suction canisters 200a and 200b); each canister being in communication with the primary blood filter system 300, discussed below.

### Primary Blood Filter System A

As indicated above, the blood processing means of the invention, i.e., primary blood filter systems, are adapted to receive aspirated autologous blood from a patient (and, as discussed below, non-autologous blood) and extract impurities therefrom.

As discussed in detail below, in a preferred embodiment, the primary filter systems comprise solely gravity-driven filter systems, which are adapted to extract impurities from the autologous and non-autologous blood without damaging the erythrocytes in the blood and, hence, inducing hemolysis of the erythrocytes in the blood and loss of seminal native blood components, including native erythrocytes, platelets, white blood cells, plasma proteins and antibodies.

As also indicated above, in some embodiments, the blood reinfusion systems of the invention solely comprise a primary blood filter system of the invention, i.e., a stand-alone blood processing system.

As further indicated above, in some embodiments, the blood reinfusion systems comprise first blood collection means, i.e., at least one suction canister of the invention, and a primary blood filter system of the invention, such as depicted in FIGS. 1 and 2, wherein filter system 300 is in communication with suction canister 200a.

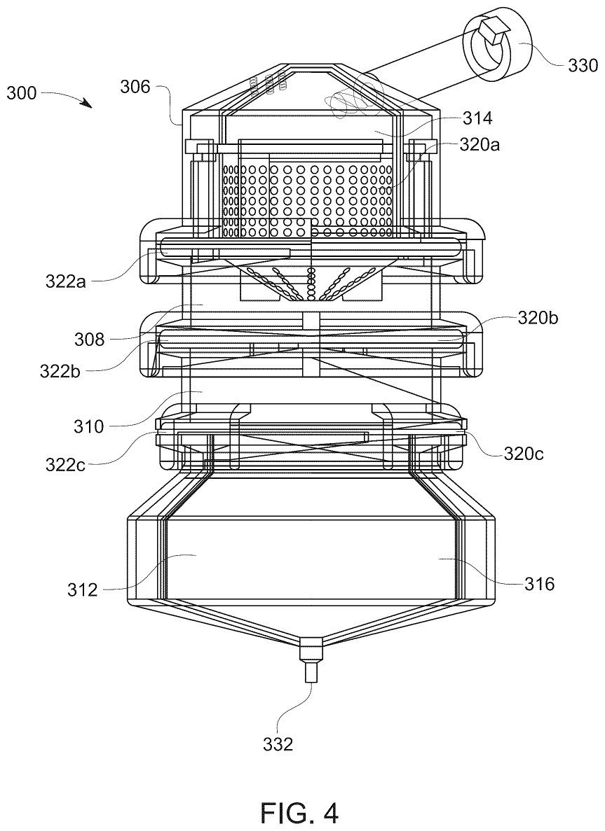

Referring now to FIG. 4, one embodiment of a primary blood filter system of the invention, i.e., primary blood filter system 300, will be described in detail.

In a preferred embodiment, filter system 300 comprises one embodiment of the filter system disclosed in priority U.S. application Ser. No. 18/220,373, which, as depicted in FIG. 4, comprises a three-stage gravity-driven filter system comprising a top housing portion 306, a first intermediate housing portion 308, a second intermediate housing portion 310, and a bottom housing portion 312; the top housing portion 306, first intermediate housing portion 308 and second intermediate housing portion 310 each comprising at least one filter.

According to the invention, the filter system 300 can also comprise a two-stage gravity-driven filter system comprising the top housing portion 306, first intermediate housing portion 308 and a bottom housing portion 312; the top housing portion 306 and first intermediate housing portion 308 similarly comprising at least one filter.

The filter system 300 can also comprise additional intermediate housing portions, such as a third and fourth intermediate housing portion, wherein each additional intermediate housing portion would similarly comprise at least one filter.

The intermediate housing portions of the filter system 300, e.g., first intermediate housing portion 308, are also referred to herein as “filter modules”.

As set forth in priority U.S. application Ser. No. 18/220,373 and depicted in FIG. 4, the top housing portion 306 comprises a first reservoir 314 that is adapted and configured to receive autologous blood therein, in this instance, the first processed autologous blood transmitted from the suction canister 200a (i.e., autologous blood with first impurities extracted therefrom), and the bottom housing portion 312 comprises a second reservoir 316 that is adapted and configured to receive processed autologous blood.

In some embodiments, the first reservoir 314 preferably comprises a volume in a range of approximately 60.0 ml to approximately 300.0 ml and the second reservoir 316 preferably comprises a volume in a range of approximately 100.0 ml to approximately 400.0 ml.

As set forth in priority U.S. application Ser. No. 18/220,373, the top housing portion 306 of the filter system 300 comprises a first filter 320a, the first intermediate housing portion 308 comprises a second filter 320b, and the second intermediate housing portion 310 comprises a third filter 320c.

According to the invention, the first, second and third filters 320a, 320b, 320c can similarly comprise any suitable pore size.

Preferably, the first filter 320a and/or second filter 320b and/or third filter 320c comprises a pore size less than approximately 10.0 mm.

Thus, in some embodiments, the first filter 320a and/or second filter 320b and/or third filter 320c comprises a pore size less than approximately 5.0 mm.

In some embodiments, the first filter 320a and/or second filter 320b and/or third filter 320c comprises a pore size in the range of approximately 3.0 mm to approximately 5.0 mm.

In some embodiments, the first filter 320a and/or second filter 320b and/or third filter 320c comprises a pore size in the range of approximately 1.0 mm to approximately 2.0 mm.

In some embodiments, the first filter 320a and/or second filter 320b and/or third filter 320c comprises a pore size in the range of approximately 40.0 micron to approximately 1000.0 micron.

In some embodiments, the first filter 320a and/or second filter 320b and/or third filter 320c comprises a pore size in the range of approximately 100.0 micron to approximately 500.0 micron.

In some embodiments, the first filter 320a and/or second filter 320b and/or third filter 320c comprises a pore size in the range of approximately 200.0 micron to approximately 300.0 micron.

In some embodiments, the first filter 320a and/or second filter 320b and/or third filter 320c comprises a pore size less than approximately 250.0 micron.

In some embodiments, the first filter 320a and/or second filter 320b and/or third filter 320c comprises a pore size in the range of approximately 170.0 micron to approximately 260.0 micron.

In some embodiments, the first filter 320a and/or second filter 320b and/or third filter 320c comprises a pore size in the range of approximately 150.0 micron to approximately 225.0 micron.

In some embodiments, the first filter 320a and/or second filter 320b and/or third filter 320c comprises a pore size in the range of approximately 50.0 micron to approximately 100.0 micron.

In some embodiments, the first filter 320a and/or second filter 320b and/or third filter 320c comprises a pore size less than approximately 50.0 micron.

In some embodiments, the first filter 320a and/or second filter 320b and/or third filter 320c comprises a pore size in the range of approximately 10.0 micron to approximately 40.0 micron.

In a preferred embodiment, the first filter 320a comprises a pore size in the range of approximately 1.0 mm to approximately 5.0 mm, more preferably, a pore size of approximately 4.0 mm, even more preferably, a pore size of approximately 2.0 mm.

In a preferred embodiment, the second filter 320b comprises a pore size in the range of approximately 40.0 micron to approximately 1000.0 micron, more preferably, a pore size less than approximately 500.0 micron, even more preferably, a pore size in the range of approximately 170.0 micron to approximately 260.0 micron.

In a preferred embodiment, the third filter 320c comprises a pore size less than 50.0 micron, more preferably, a pore size in the range of approximately 10.0 micron to approximately 40.0 micron, even more preferably, a pore size of approximately 40.0 micron.

In a preferred embodiment of the invention, the first filter 320a is configured and adapted to receive the first processed autologous blood from the suction canister 200a and isolate and extract first excess, i.e. remaining, impurities from the first processed autologous blood, whereby second processed autologous blood is obtained, the second filter 320b is adapted to receive the second processed autologous blood from the first filter 320a and isolate and extract second excess impurities from the second processed autologous blood, whereby third processed autologous blood is obtained, and the third filter 320c is adapted to receive the third processed autologous blood from the second filter 320b and isolate and extract third excess impurities from the third processed autologous blood.

Applicant has found that, when the autologous blood (and non-autologous blood) is processed with the gravity-driven blood filter system 300 described above, purified whole autologous blood (and non-autologous) is obtained.

Indeed, Applicant has found that, when autologous (and non-autologous) blood is processed with the gravity-driven blood filter system 300 described above, the processed blood retains and, hence, comprises all seminal native blood components; specifically, erythrocytes, platelets, white blood cells, plasma proteins and antibodies.

Applicant has also found that the processed blood also exhibits and, hence, retains the following native blood components: bilirubin, aspartate aminotransferase (AST), alanine transaminase (ALT), albumin, serum potassium (K) and serum calcium (CA).

Applicant has additionally found that the retained content (or concentration) of the noted blood components in the processed blood is at least 98.0%.

Applicant has additionally found that the hematocrit percent (Hct %) of the processed blood is comparable to the Hct % of the pre-processed blood.

Applicant has additionally found that the integrity of native erythrocytes in the processed blood is uncompromised.

Applicant has additionally found that the processed whole blood is substantially devoid of impurities, including blood clots, emboli, tissue debris, foreign particles, etc. Indeed, the impurity content in the whole blood is less than 0.05%.

Applicant has additionally found that blood flow through the blood filter assembly 300 comprises a flow rate of at least 500.0 cc/min.

According to the invention, the first, second and third filters 320a, 320b, 320c can comprise any acceptable surgical material, e.g., stainless steel, and form. In a preferred embodiment, the first filter 320a comprises a perforated filter and the second and third filter 320b, 320c comprise mesh filters.

In some embodiments of the invention, the first intermediate portion 308 or the second intermediate portion 310 of the filter system 300 comprises or includes a membrane filter, comprising a pore size in the range of approximately 0.0001 micron to approximately 100.0 micron.

In some embodiments of the invention, the second intermediate portion 310 of the filter system 300 comprises or includes an emboli filter adapted to remove residual air, if any, from the processed autologous blood.

In a preferred embodiment, the top housing portion 306, first intermediate housing portion 308, and second intermediate housing portion 310 are detachably coupled in succession, whereby the autologous blood is successively filtered through filters 320a, 320b, and 320c.

As indicated above, in a preferred embodiment, the filter system 300 is solely gravity-driven, i.e., filtering of the autologous blood through filters 320a, 320b, and 320c is achieved solely by the force of gravity.

According to the invention, in some embodiments, the filter system 300 can further comprise means for providing negative pressure therein, wherein the autologous blood is successively filtered and, hence, processed through filters 320a, 320b, and 320c via the negative pressure provided in the blood filter assembly 300.

In a preferred embodiment, the top housing portion 306, the first intermediate housing portion 308, the second intermediate housing portion 310, and the bottom housing portion 312 are readily detachable from one another for case of access and cleaning of the respective housing portions and cleaning and replacing the filters 320a, 320b, and 320c.

As set forth in priority U.S. application Ser. No. 18/220,373 and depicted in FIG. 4, the top housing portion 306 of the blood filter assembly 300 comprises an inlet port 330 adapted to receive the blood filter inlet line 302 and, hence, first processed autologous blood from the suction canister 200a transmitted therethrough.

In a preferred embodiment, the inlet port 330 comprises a luer connector to facilitate releasable connection of the suction canister 200a to the blood filter assembly 300.

As depicted in FIG. 4, the bottom housing portion 312 of the blood filter assembly 300 further comprises an outlet port 332, which is adapted to receive the outlet line 305 of the filter system 300 to facilitate transfer of the autologous blood processed by the filter system 300, i.e., fourth processed autologous blood, to the blood collection container 400a.

In a preferred embodiment, the outlet port 332 similarly comprises a luer connector to facilitate releasable connection of the filter system 300 to the blood collection container 400a.

As further set forth in priority U.S. application Ser. No. 18/220,373, in some embodiments, the inner walls of the top housing portion 306 comprise channels that allow for the first filter 320a, when inserted into the top housing portion 306 from the bottom of the top housing portion 306 (when the top housing portion 306 is detached from the first intermediate housing portion 308), to be twisted in a first direction and be locked in place, and twisted in a second direction (opposite to the first direction) to unlock.

As also set forth in priority U.S. application Ser. No. 18/220,373, in some embodiments, a flow redirector element is positioned above and proximate each of the filters 320a, 320b, 320c, to bias and control the blood flow thereto, e.g., blood flow towards a side or portion of the filters. By configuring the flow redirector element in such manner, impurity accumulation is focused to a portion of the filters while the remaining portion(s) of the filters remains open and unobstructed.

According to the invention, the plane of the flow redirector element can be inclined at any desired predefined angle, e.g., 30.0 degrees to 45.0 degrees from a horizontal plane.

In some embodiments, the predefined angle of the flow redirector element ranges from approximately 0.0 degrees to approximately 60.0 degrees from the horizontal plane.

As further set forth in priority U.S. application Ser. No. 18/220,373 and depicted in FIG. 4, the top housing portion 306, first intermediate housing portion 308, second intermediate housing portion 310, and bottom housing portion 312 of the filter system 300 are sealed, when connected, via a plurality of gaskets 322a, 322b, 322c and O-rings.

Further features and embodiments of the filter system 300 are set forth in U.S. application Ser. No. 18/220,373, which is expressly incorporated by reference herein in its entirety.

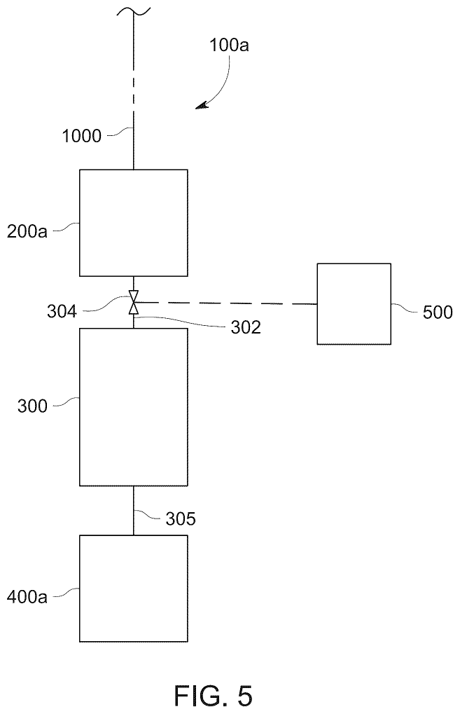

Referring now to FIG. 5, in some embodiments of the invention, the inlet line (i.e., conduit means) 302 of the filter system 300 (see also FIG. 1) comprises a valve assembly 304, which is adapted to modulate blood flow from the suction canister 200a into the filter system 300.

According to the invention, the valve assembly 304 can comprise any suitable valve assembly including, without limitation, a passive (one-way) valve assembly, an active valve assembly and a multi-way valve assembly.

As depicted in FIG. 5, in the noted embodiments, the blood reinfusion system 100a further comprises control means 500, which is programmed to control the valve assembly 304 and, hence, blood flow into the filter system 300.

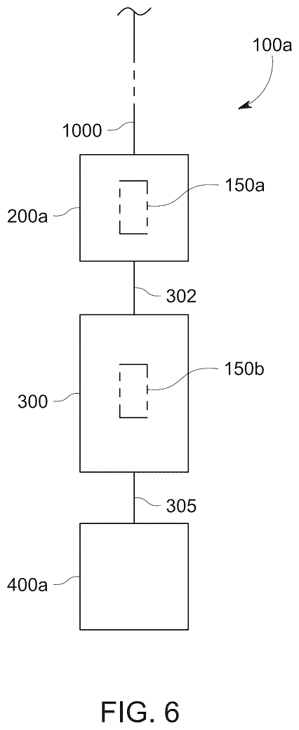

Referring now to FIG. 6, in some embodiments of the invention, the filter system 300 further comprises a second sensor system 150b, which is adapted to monitor blood flow through the filter system 300.

In some embodiments of the invention, the top housing portion 306 of the filter system 300 further comprises an agent inlet configured and adapted to deliver blood processing agents and compositions into the filter system 300, when it is desired to mix such agents and/or compositions with the autologous blood. In some embodiments, the blood processing agents and compositions are pre-loaded in the top housing portion 306 and/or bottom housing portion 312 of the filter system 300 in a powdered or lyophilized form.

Exemplar blood processing agents and compositions include, without limitation, anticoagulants, such as heparin or coumadin; thrombolytics, such as tissue plasminogen activator (tPA), streptokinase, or urokinase; and hormones, such as erythropoietin (EPO). Further agents and compositions are disclosed in Applicant's U.S. application Ser. No. 18/638,483, which is incorporated by reference herein in its entirety.

### Blood Collection Container(s)

As indicated above, the blood collection containers of the invention are configured and adapted to receive and contain the processed autologous blood from the filter systems of the invention.

According to the invention, the blood collection containers can comprise any configuration and size. In one preferred embodiment, the blood collection containers comprise a blood collection (or transfer) bag, such as a blood transfer or transfusion bag, to facilitate reinfusion of the processed autologous blood into a patient.

In the noted preferred embodiment, the blood collection bag preferably comprises a size or capacity in the range of approximately 200.0 ml to approximately 1000.0 ml.

In a preferred embodiment, the blood collection containers of the invention are also configured and adapted to receive blood processing agents and compositions, including, without limitation, the aforementioned blood processing agents and compositions, therein.

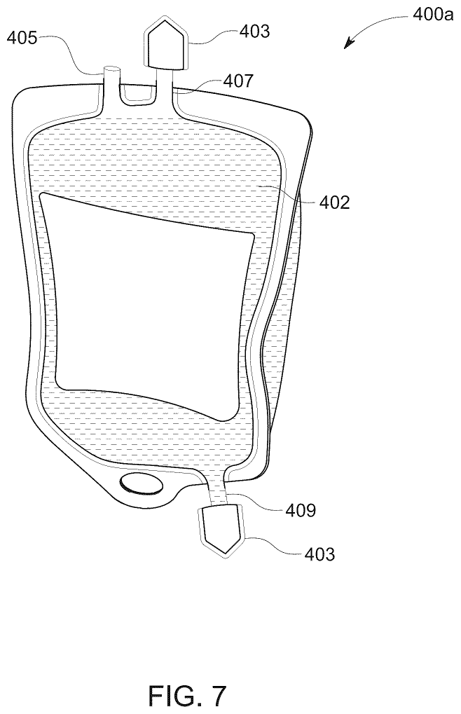

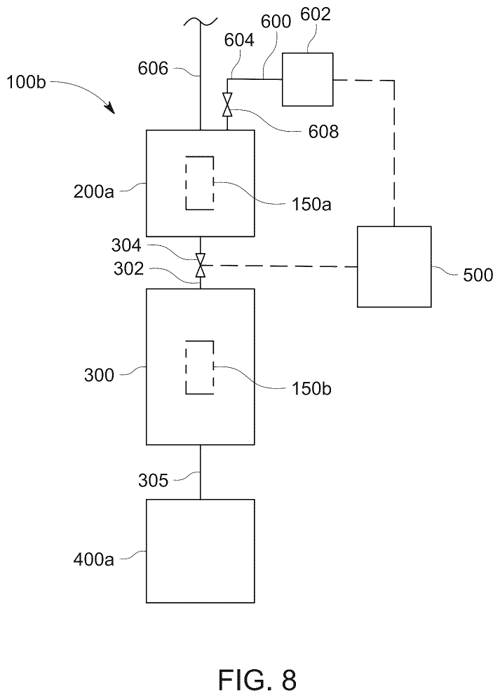

Referring now to FIG. 7, there is depicted one embodiment of a blood collection container of the invention in the form of a blood collection bag.

As depicted in FIG. 7, the blood collection container, i.e., bag, 400a comprises a scaled pouch comprising a blood inlet 405, which is sized and adapted to receive the outlet line 305 of the filter system 300 (see FIG. 2) and, hence, processed autologous blood (denoted “402”) from the filter system 300, an air vent 407, and a blood outlet 409 that is sized and adapted to receive a blood transfusion line to reinfuse the processed autologous blood into the patient.

As further depicted in FIG. 7, the air vent 407 and blood outlet 409 are further adapted to receive end caps 403, which are sized and adapted to close and seal the air vent 407 and blood outlet 409 when appropriate.

In some embodiments, one or more of the aforementioned blood processing agents and compositions are pre-loaded in the blood collection container 400a in a powdered or lyophilized form.

As indicated above, the blood reinfusion system 100a can further comprise two (2) blood collection containers; each adapted to couple to the blood filter assembly 300.

As indicated above, according to the invention, the blood reinfusion system 100a can also comprise a modular system, wherein the suction canister 200a and blood filter assembly 300 are detachably coupled and thus the blood filter inlet line 302 of the blood filter assembly 300 is eliminated, or the suction canister 200a, filter system 300 and blood collection container 400a are detachably coupled and thus the blood filter inlet line 302 and blood filter outlet line 305 of the filter system 300 are eliminated.

According to the invention, the noted modular systems can further comprise on-off switches at the interconnections between the suction canister 200a and filter system 300, and the filter system 300 and bag(s) 400a, if part of the modular system.

Referring now to FIG. 8, there is depicted a schematic illustration of a further embodiment of a blood reinfusion system of the invention (denoted “100b”).

As depicted in FIG. 8, the blood reinfusion system 100b similarly comprises suction canister 200a, filter system 300, and blood collection container 400a, discussed above. The blood reinfusion system 100b further comprises the control means 500.

As further depicted in FIG. 8, the blood reinfusion system 100b further comprises aspiration means 600, comprising a negative pressure (or suction) line 604, which is sized and configured to engage and, hence, communicate with the suction inlet 210 of the suction canister 200a, an aspiration catheter 606 adapted to be positioned proximate a surgical site of a patient, means for providing negative pressure and, hence, a suction force, though the aspiration catheter 606, and control means 500 for controlling the negative pressure means.

In a preferred embodiment, the negative pressure means, i.e., means for providing the suction force though the aspiration catheter 606, comprises a conventional pump assembly 602.

In a preferred embodiment, the pump assembly 602 is configured and adapted to generate and provide a negative pressure in the suction canister 200a via negative pressure line 604, which provides the suction force though the aspiration catheter 606 connected thereto.

In a preferred embodiment, the pump assembly 602 is configured and adapted to provide a negative pressure up to −400.0 mm Hg.

As further depicted in FIG. 8, in some embodiments, the aspiration means 600 further comprises a valve assembly 608, which is disposed in the negative pressure line 604. In the noted embodiments, the valve assembly 608 is adapted to modulate the negative pressure transmitted to the suction canister 200a and, hence, is also in communication with the control means 500 of the system 100b, which is additionally programmed to control the valve assembly 608 and, hence, negative pressure transmitted to the suction canister 200a.

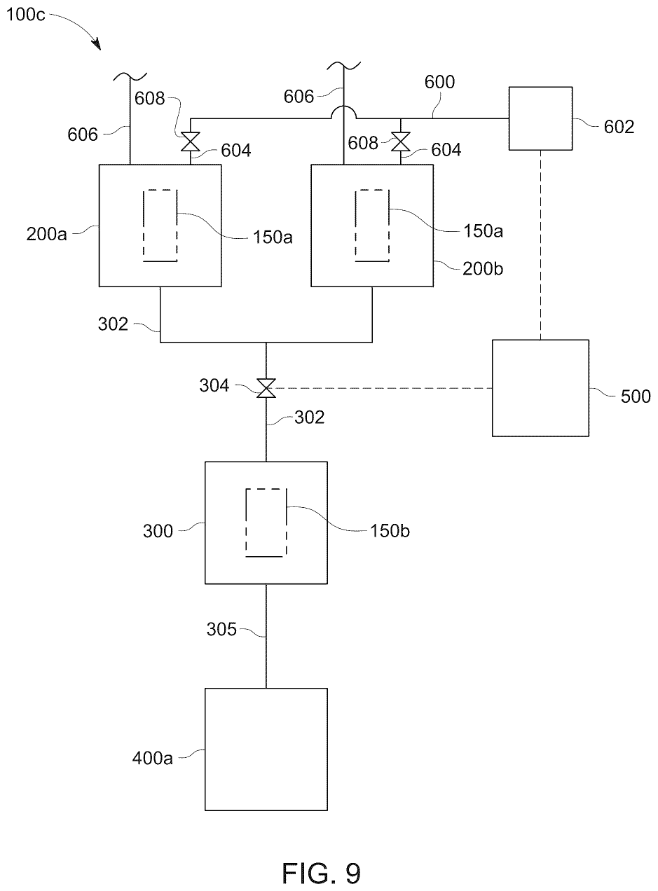

Referring now to FIG. 9, there is depicted a schematic illustration of a further embodiment of a blood reinfusion system of the invention (denoted “100c”).

As depicted in FIG. 9, the blood reinfusion system 100c similarly comprises the suction canister 200a, filter system 300, blood collection container 400a, control means 500 and aspiration means 600 depicted in FIG. 8 discussed above.

However, as depicted in FIG. 9, the blood reinfusion system 100c further comprises a second suction canister (denoted “200b”), which, according to the invention, is substantially similar in construction, size and function as suction canister 200a described above.

As depicted in FIG. 9, the second suction canister 200b is similarly in communication with the aspiration means 600 via negative pressure line 604, as described above, and filter system 300 via blood inlet line 302.

According to the invention, blood reinfusion systems 100b and 100c can also comprise modular systems, such as the modular blood reinfusion system 100a, described above.

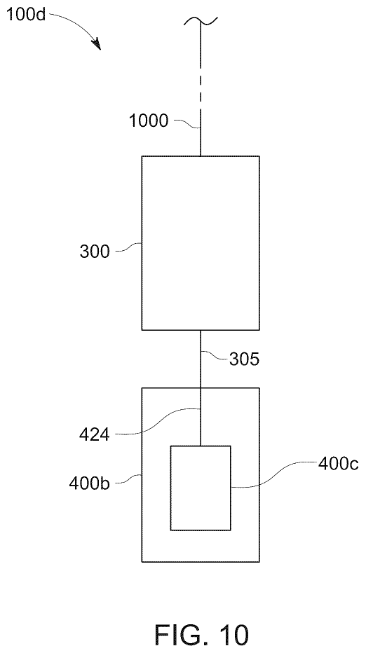

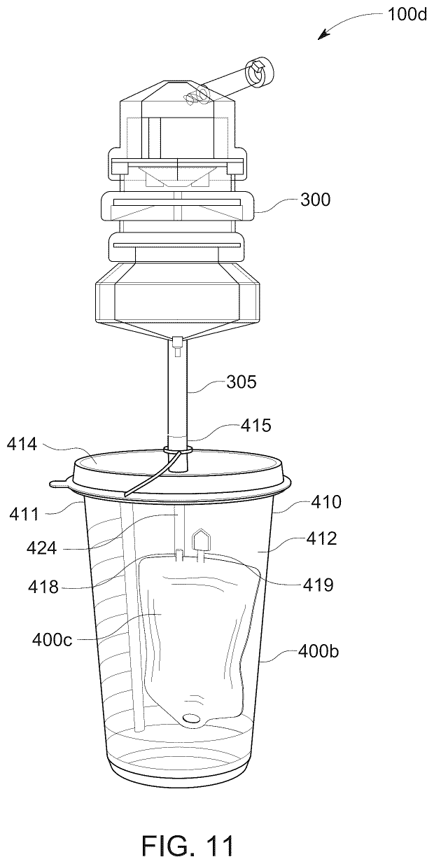

Referring now to FIGS. 10 and 11, there is depicted a schematic illustration of a further embodiment of a blood reinfusion system of the invention (denoted “100d”).

As depicted in FIGS. 10 and 11, the blood reinfusion system 100d similarly comprises the three-stage filter system 300, discussed above. The blood reinfusion system 100d further comprises a unique blood collection container 400b.

As depicted in FIG. 11, the blood collection container 400b comprises an outer container 410, comprising an inner fluid reservoir 412 and a top cap 414, which, according to the invention, is similarly sized and configured to sealably engage the top open portion 411 of the outer container 410.

As further depicted in FIG. 11, the blood collection container 400b further comprises an inner blood collection container or bag 400c, which is disposed in the inner fluid reservoir 412 of the outer container 410.

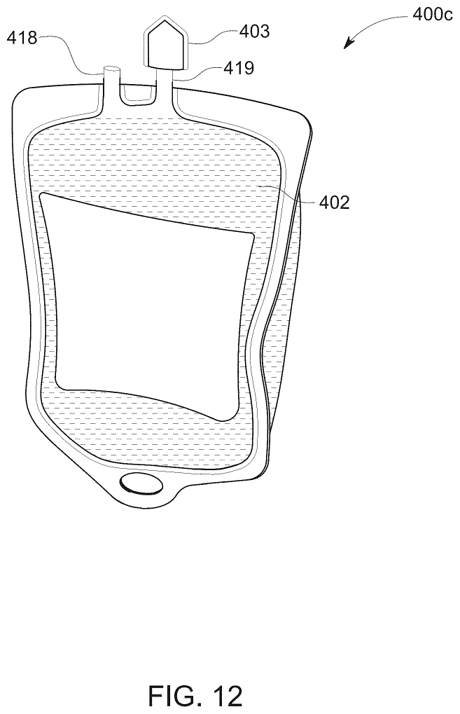

As additionally depicted in FIGS. 11 and 12, the inner blood collection bag 400c similarly comprises a sealed pouch comprising a blood inlet 418, which is sized and configured to receive the blood inlet line 424 of the bag 400c, and an air vent or filter 419, which is similarly sized and adapted to receive an end cap 403 when appropriate.

To facilitate communication of the inner blood collection bag 400c with the filter system 300 and, hence, receipt of processed blood therefrom (denoted “402” in FIG. 12), in a preferred embodiment, the top cap 414 of the blood collection container 400b comprises a blood inlet 415, which is sized and configured to receive the outlet line 305 of the filter system 300 and the blood inlet line 424 of the inner blood collection bag 400c.

To facilitate the noted communication of the blood inlet 415 of the container cap 414 with the blood inlet line 424 of the inner blood collection bag 400c, the blood inlet 415 preferably extends into the inner fluid reservoir 412 of the outer container 410 when the top cap 414 is engaged thereto.

According to the invention, blood flow into and through the filter system 300 and, thereby into the inner blood collection bag 400c is facilitated by the negative pressure (or vacuum) of the external aspiration system and, hence, catheter 1000 (and, hence, aspiration system). In addition to the processed blood transmitted through the filter system 300, the inner blood collection bag 400c thus may, and, in all likelihood will, contain undesirable air.

However, according to the invention, when the external aspiration catheter 1000 is disconnected from the blood filter 300 (and, hence, the negative pressure in the system 100d is released), the inner blood collection bag 400c relaxes and, hence, contracts, and the air in the bag 400c is released via air vent 419 when unsealed.

In a preferred embodiment, the outer container 410 of the blood collection container 400b comprises a rigid structure, such as, by way of example, a polypropylene housing or case, which secures the inner blood collection bag 400c in a sealed, sterile protective structure.

According to the invention, the outlet line 305 of the filter system 300 can similarly comprise a valve assembly, such as valve assembly 304 depicted in FIG. 9, to modulate blood flow into the blood collection container 400b.

According to the invention, one or more of the aforementioned agents and compositions, e.g., anticoagulants, can similarly be pre-loaded into the blood collection bag 400c in a powdered or lyophilized form.

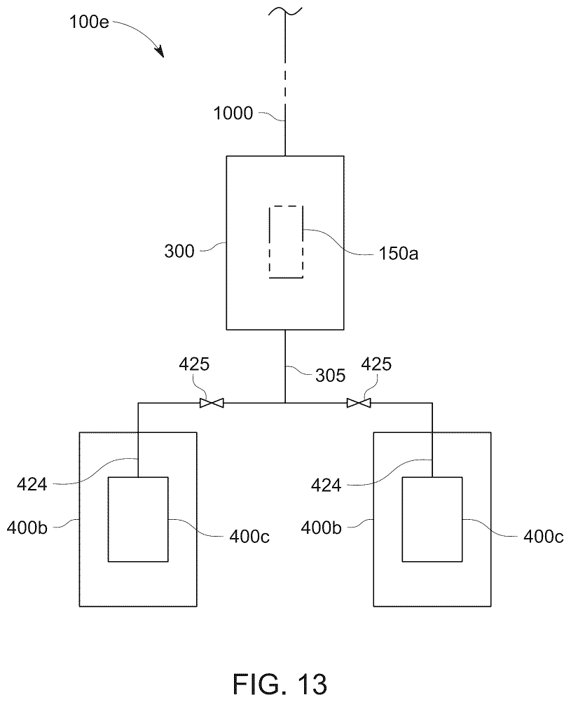

Referring now to FIG. 13, there is depicted a schematic illustration of a further embodiment of a blood reinfusion system of the invention (denoted “100e”).

As depicted in FIG. 13, the blood reinfusion system 100e is similar to blood reinfusion system 100d depicted in FIG. 10 and discussed above, except, in this embodiment, the blood reinfusion system 100e comprises two blood collection containers 400b.

As further depicted in FIG. 13, each blood collection container 400b is in fluid communication with the filter system 300 via blood filter outlet line 305.

According to the invention, valve assemblies 425 can be disposed in the blood outlet line 305 proximate each blood collection container 400b to modulate blood flow into the containers 400b. In such embodiments, the blood reinfusion system 100e would further comprise control means programmed and configured to control the valve assemblies, such as control means 500 depicted in FIG. 5 and described above.

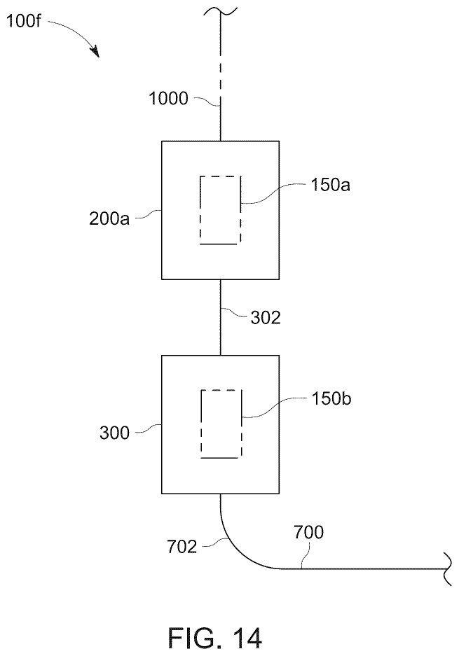

Referring now to FIG. 14, there is depicted a schematic illustration of a further embodiment of a blood reinfusion system of the invention (denoted “100f”).

As depicted in FIG. 14, the blood reinfusion system 100f similarly comprises the suction canister 200a and filter system 300 of the base blood reinfusion system 100a depicted in FIGS. 1 and 2.

However, as further depicted in FIG. 14, the blood reinfusion system 100f further comprises patient blood infusing means (denoted “700”) adapted and configured to continuously reinfuse the processed and, hence, purified whole autologous blood into a patient during processing via the filter system 300 of the system 100f.

According to the invention, the purified whole autologous blood is reinfused into the patient via a transfusion line (i.e., conduit means) 702, which is connected directly to the outlet line 305 of the filter system 300.

According to the invention, the blood reinfusion system 100d can also comprise a modular system, wherein the filter system 300 and blood collection container 400b are interconnected and thus the outlet line 305 of the filter system 300 is eliminated.

According to the invention, the noted modular systems can similarly further comprise an on-off switch at the interconnection between the filter system 300 and blood collection container 400b.

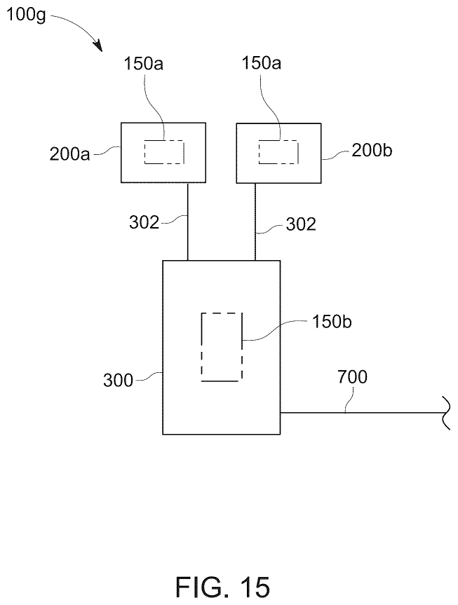

Referring now to FIG. 15, there is depicted a schematic illustration of a further embodiment of a blood reinfusion system of the invention (denoted “100g”).

As illustrated in FIG. 15, the blood reinfusion system 100g similarly comprises filter system 300 and the patient blood infusing means 700 of blood reinfusion system 100f depicted in FIG. 14 and discussed above.

As further illustrated in FIG. 15, the blood reinfusion system 100f comprises a plurality of suction canisters; preferably, suction canisters 200a and 200b, described above.

### Primary Blood Filter System B

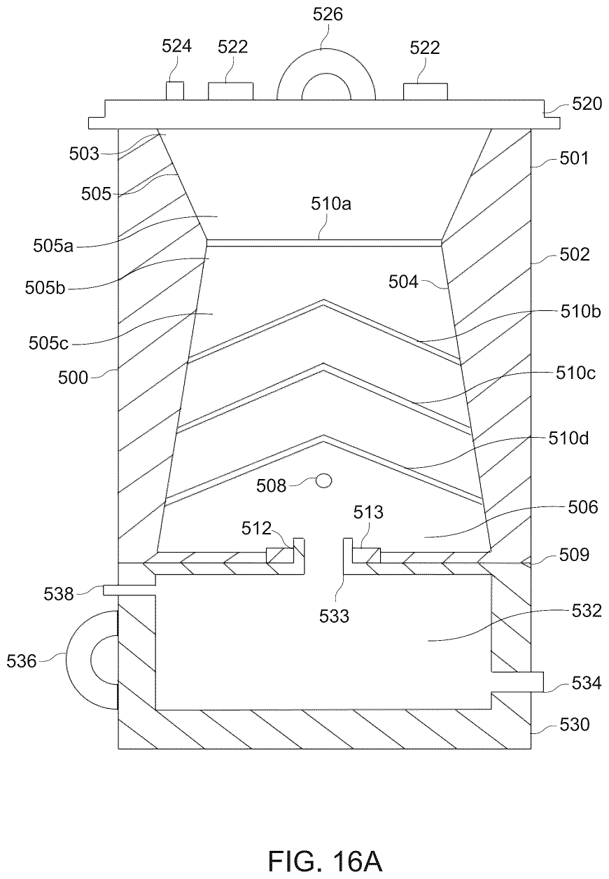

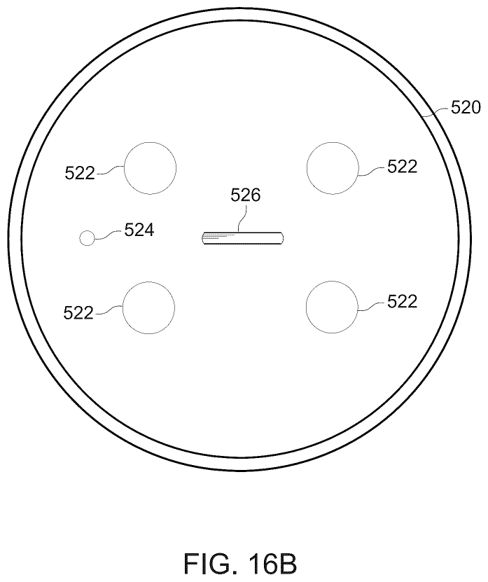

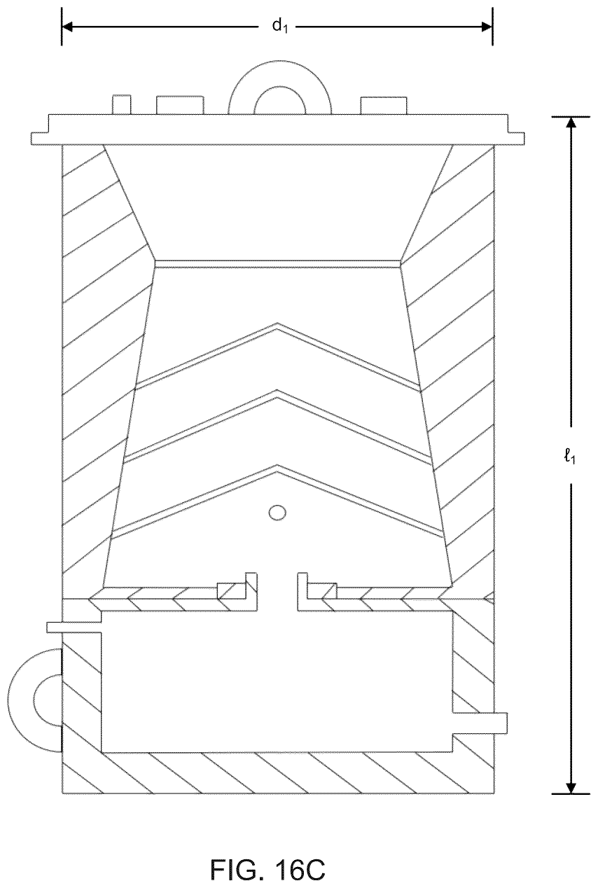

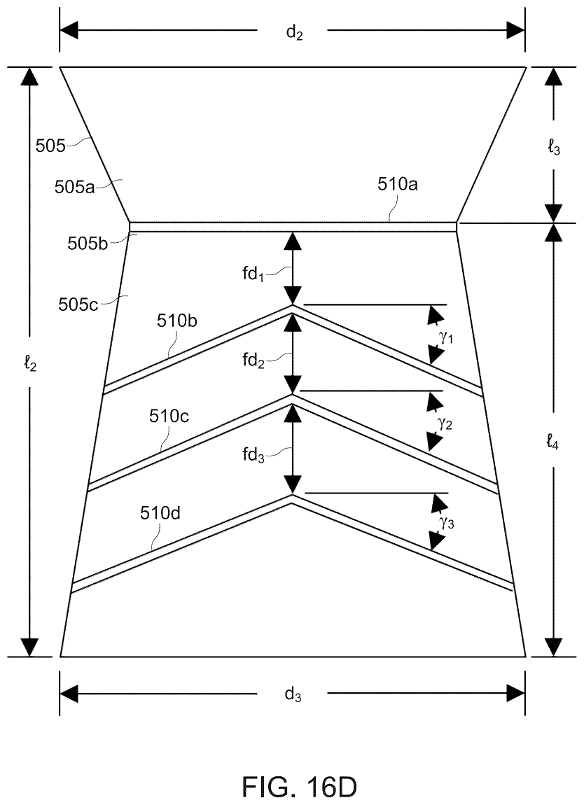

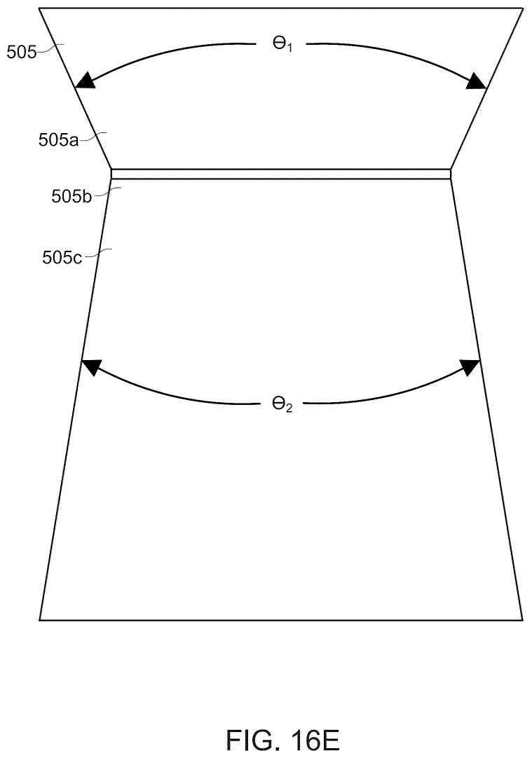

Referring now to FIGS. 16A and 16B, there is depicted a further embodiment of a primary blood filter system of the invention (denoted “500”).

According to the invention, the filter system 500 can be substituted for filter system 300 and, hence, employed in blood reinfusion systems 100a-100g, described above.

According to the invention, filter system 500 can also be employed as a stand-alone blood processing system.

As illustrated in FIG. 16A, the filter system 500 comprises a blood processing canister 501 comprising an outer housing 502. In a preferred embodiment, the housing 502 comprises an internal fluid passageway 503 and a detachably coupled blood collection container 530.

According to the invention, the internal fluid passageway 503 is sized and configured to receive autologous (and non-autologous) blood therein and allow the blood to be transmitted therethrough.

As illustrated in FIG. 16A, in a preferred embodiment, the internal fluid passageway 503 comprises a venturi region 505, which, as discussed in detail below, is a seminal feature of the blood reinfusion system 500.

As further illustrated in FIG. 16A, the venturi region 505 comprises an inlet cone region 505a, a restricted flow path region 505b and an outlet cone region 505c.

According to the invention, the inlet cone region 505a is adapted to receive autologous blood transmitted to the filter system 500 and, hence, defines a top blood collection chamber or reservoir.

Referring now to FIGS. 16C-16E, the dimensional parameters and relationships therebetween of the filter system 500 will be described in detail.

Referring first to FIG. 16C, in some embodiments, the length ( [symbol] ₁) of the filter system 500 (with a 500 cc blood collection chamber 532, which is discussed below) is preferably in the range of approximately 19.0 cm to approximately 25.0 cm.

In a preferred embodiment, the total length ( [symbol] ₁) of the filter system 500 is in the range of approximately 19.0 cm to approximately 21.0 cm.

In some embodiments, the diameter of the outer housing 502 (denoted “d₁”) of the system housing 500 is preferably in the range of approximately 10.0 cm to approximately 15.0 cm.

In a preferred embodiment, the diameter of the outer housing 502 (d₁) of the system filter 500 is approximately 12.5 cm.

Referring now to FIGS. 16D and 16E, in some embodiments, the inlet diameter of the venturi inlet cone region 505a (denoted “d₂”) is preferably in the range of approximately 9.0 cm to approximately 14.0 cm.

In a preferred embodiment, the length of the venturi region 505 (denoted “ [symbol] ₂”) is in the range of approximately 15.0 cm to approximately 17.0 cm.

In some embodiments, the cone angle of the venturi inlet cone region 505a (denoted “θ₁”) is preferably in the range of approximately 26.0° to approximately 66.0°.

In a preferred embodiment, the cone angle of the venturi inlet cone region 505a (θ₁) is in the range of approximately 36.0° to approximately 56.0°.

In some embodiments, the length of the venturi inlet cone region 505a (denoted “ [symbol] ₃”) is preferably approximately 20.0% to approximately 50.0% of total length of venturi region 505 ( [symbol] ₂).

In a preferred embodiment, the length of the venturi inlet cone region 505a ( [symbol] ₃) is approximately 25.0% to approximately 35.0% of total length of venturi region 505 ( [symbol] ₂).

In some embodiments, the cone angle of the venturi outlet cone region 505c (denoted “θ₂”) is preferably in the range of approximately 10.0° to approximately 60.0°.

In a preferred embodiment, the cone angle of the venturi outlet cone region 505c (θ₂) is in the range of approximately 10.0° to approximately 45.0°.

In some embodiments, the length of the venturi outlet cone region 505c (denoted “ [symbol] ₄”) is preferably approximately 70.0% to approximately 90.0% of total length of venturi region 505 ( [symbol] ₂).

In a preferred embodiment, the length of the venturi outlet cone region 505c ( [symbol] ₄) is approximately 75.0% to approximately 85.0% of total length of venturi region 505 ( [symbol] ₂).

In some embodiments, the outlet diameter of the venturi outlet cone region 505c (denoted “d₃”) is preferably in the range of approximately 9.0 cm to approximately 14.0 cm.

As further illustrated in FIG. 16A, the venturi region 505 further comprises a gravity-driven blood filtering system 510.

In some embodiments of the invention, the gravity-driven blood filtering system 510 comprises at least one filter adapted to extract impurities from the autologous blood.

In some embodiments, the filter comprises a pore size less than approximately 200.0 microns.

As illustrated in FIG. 16A, in a preferred embodiment of the invention, the gravity-driven blood filtering system 510 comprises a plurality of integrated filters, i.e., filters 510a, 510b, 510c and 510d.

According to the invention, filters 510a, 510b, 510c and 510d can comprise any suitable pore size, including, without limitation, the filter pore sizes referenced above.

In a preferred embodiment of the invention, the first filter, i.e. filter 510a, comprises a pore size less than approximately 10.0 mm, more preferably, a pore size in the range of approximately 3.0 mm to approximately 5.0 mm, the second filter, i.e. filter 510b, comprises a pore size less than approximately 5.0 mm, more preferably, a pore size in the range of approximately 1.0 mm to approximately 3.0 mm, the third filter, i.e. filter 510c, comprises a pore size less than approximately 300.0 micron, more preferably, a pore size in the range of approximately 100.0 micron to approximately 200.0 micron, even more preferably, a pore size in the range of approximately 140.0 micron to approximately 160.0 micron, and the fourth filter, i.e. filter 510d, comprises a pore size less than approximately 100.0 micron, more preferably, a pore size in the range of approximately 30.0 micron to approximately 60.0 micron.

In a preferred embodiment of the invention, filter 510a is configured and adapted to receive initially processed autologous blood from at least one suction canister, e.g., suction canister 200a, and isolate and extract first excess, i.e. remaining, impurities from the initially processed autologous blood, whereby first processed autologous blood is obtained, filter 510b is adapted to receive the first processed autologous blood from filter 510a and isolate and extract second excess impurities from the first processed autologous blood, whereby second processed autologous blood is obtained, filter 510c is adapted to receive the second processed autologous blood from filter 510b and isolate and extract third excess impurities from the second processed autologous blood, whereby third processed autologous blood is obtained, and filter 510d is adapted to receive the third processed autologous blood from filter 510c and isolate and extract fourth excess impurities from the third processed autologous blood, whereby purified whole autologous blood is similarly obtained.

Indeed, Applicant has found that, when the autologous blood (and non-autologous blood) is transmitted through the internal fluid passageway 503 and, hence, venturi region 505 of the gravity-driven blood filter system 500 described above, the blood exhibits a substantially laminar flow pattern in and thorough the venturi outlet cone region 505c, whereby the risk of shear forces imparted to blood and, hence, induced hemolysis of the erythrocytes in the blood is abated.

In a preferred embodiment, when autologous (and non-autologous) blood is processed with the gravity-driven blood filter system 500, the processed blood similarly retains and, hence, comprises all seminal native blood components; specifically, native erythrocytes, platelets, white blood cells, plasma proteins and antibodies.

In a preferred embodiment, the processed blood also exhibits and, hence, retains the following native blood components: bilirubin, aspartate aminotransferase (AST), alanine transaminase (ALT), albumin, serum potassium (K) and serum calcium (CA).

Indeed, in a preferred embodiment, the retained content (or concentration) of the noted blood components in the processed blood is at least 98.0%.

In a preferred embodiment, the hematocrit percent (Hct %) of the processed blood is also comparable to the Hct % of the pre-processed blood.

In a preferred embodiment, the integrity of native erythrocytes in the processed blood is uncompromised, i.e., the erythrocytes in the processed blood are not damaged.

In a preferred embodiment, the impurity content in the processed whole blood is less than 0.05%.

In a preferred embodiment, blood flow through the internal fluid passageway 503 and, hence, gravity-driven blood filtering system 510 similarly comprises a flow rate of at least 500.0 cc/min.

According to the invention, filters 510b, 510c and 510d can also comprise a membrane filter, comprising a pore size in the range of approximately 0.0001 micron to approximately 100.0 micron.

Referring back to FIG. 16D, in some embodiments of the invention, the distance between the first filter, i.e., filter 510a, and the second filter, i.e., filter 510b, is preferably in the range of approximately 30.0 mm to approximately 50.0 mm, more preferably, approximately 35.0 mm to approximately 45.0 mm.

In a preferred embodiment, the distance between filter 510a and filter 510b is approximately 30.0 mm.

In some embodiments of the invention, the distance between filter 510b, and the third filter, i.e., filter 510c, is preferably in the range of approximately 20.0 mm to approximately 40.0 mm, more preferably, approximately 20.0 mm to approximately 35.0 mm.

In a preferred embodiment, the distance between filter 510b and filter 510c is similarly approximately 30.0 mm.

In some embodiments of the invention, the distance between filter 510c, and the fourth filter, i.e., filter 510d, is similarly preferably in the range of approximately 20.0 mm to approximately 40.0 mm, more preferably, approximately 20.0 mm to approximately 35.0 mm.

In a preferred embodiment, the distance between filter 510c and filter 510d is similarly approximately 30.0 mm.

According to the invention, filters 510a, 510b, 510c and 510d can also comprise any configuration. In some embodiments, filter 510a comprises a substantially planar configuration, such as depicted in FIG. 16A, or a domed (or convex) shape.

As depicted in FIG. 16A, in a preferred embodiment, filters 510b, 510c and 510d comprise a conical shape.

Referring again to FIG. 16D, in some embodiments of the invention, the cone angle of filter 510b (denoted “γ₁”) is preferably in the range of approximately 15.0° to approximately 45.0°, more preferably, approximately 20.0° to approximately 40.0°.

In a preferred embodiment, the cone angle (γ₁) of filter 510c is in the range of approximately 25.0° to approximately 35.0°.

In some embodiments of the invention, the cone angle of filter 510c (denoted “γ₂”) is similarly preferably in the range of approximately 15.0° to approximately 45.0°, more preferably, approximately 20.0° to approximately 40.0°.

In a preferred embodiment, the cone angle (γ₂) of filter 510c is similarly in the range of approximately 25.0° to approximately 35.0°.

In some embodiments of the invention, the cone angle of filter 510d (denoted “γ₃”) is similarly preferably in the range of approximately 15.0° to approximately 45.0°, more preferably, approximately 20.0° to approximately 40.0°.

In a preferred embodiment, the cone angle (γ₃) of filter 510d is similarly in the range of approximately 25.0° to approximately 35.0°.

As depicted in FIG. 16A, in a preferred embodiment, filters 510b, 510c and 510d comprise increasingly larger surface areas, i.e., the surface area of filter 510c is larger than the surface area of filter 510b and the surface area of filter 510d is larger than the surface area of filter 510c, to compensate for the decreased flow rate of the blood through the decreasing pore sizes of filters 510b, 510c and 510d.

In a preferred embodiment, filters 510a, 510b, 510c and 510d are removably attached to the inner wall 504 of the system housing 502 to facilitate removal for cleaning and replacement, if necessary.

In some embodiments, the blood processing canister 501 thus further comprises a removable bottom 509 that facilitates access to filters 510b, 510c and 510d.

Referring back to FIG. 16A, the filter system 500 further comprises an outlet 512 that is in communication with the venturi outlet cone region 505c (or bottom blood collection chamber) 506 of the system 500.

In a preferred embodiment, the outlet 512 is sized and adapted to receive the inlet 533 of the blood collection container 530, whereby the venturi outlet cone region 505c of the filter system 500 is in communication with the internal chamber 532 of the blood collection container 530 when coupled thereto.

In a preferred embodiment, the outlet 512 of the filter system 500 comprises a one-way valve 513 that is adapted to allow blood flow into and through the outlet 512 when the blood collection container 530 is coupled thereto and automatically close and seal the outlet 512 when the blood collection container 530 is decoupled therefrom.

As further depicted in FIG. 16A, in a preferred embodiment, the filter system 500 further comprises a one-way air vent/filter 508 disposed below filter 510d that is configured and adapted to release air out of the venturi outlet cone region 505c of the filter system 500.

As further depicted in FIG. 16A, the filter system 500 further comprises a top cap 520, which, according to the invention, is similarly sized and configured to sealably engage the blood processing canister 501.

Referring now to FIG. 16B, in a preferred embodiment, the cap 520 comprises a plurality of inlets 522 configured and adapted to couple to and, hence, facilitate communication with suction canisters of the invention, such as suction canisters 200a and 200b depicted in FIG. 15.

Referring back to FIG. 16A, the cap 520 also preferably comprises a hook 526 that is sized and configured to facilitate connection of the filter system 500 to a system stand (not shown).

As further depicted in FIG. 16A, in a preferred embodiment, the blood collection container 530 comprises an outlet 534 that is configured and adapted to couple to and, hence, communicate with a blood transfusion system and, hence, blood transfusion line associated therewith, and an air vent/filter 538.

According to the invention, the blood collection container 530 is also configured and adapted to receive the aforementioned processing agents and compositions, e.g., thrombolytics, therein, when it is desired to mix such agents and/or compositions with the processed autologous blood.

According to the invention, the internal chamber 532 of the blood collection container 530 can similarly comprise any suitable internal volume. In a preferred embodiment, the internal chamber 532 of the blood collection container 530 similarly comprises a volume of at least 500.0 cc.

As depicted in FIG. 16A, in a preferred embodiment, the blood collection container 530 further comprises a hook 536 that is sized and configured to facilitate connection of the blood collection container 530 to a blood/plasma infusion stand.

According to the invention, the filter system 500 can further comprise sensor means adapted to monitor the flow of autologous blood through the internal fluid passageway 503 and, hence, gravity-driven filtering system 510 and/or volume of processed autologous blood disposed in the internal chamber 532 of the blood collection container 530.

According to the invention, the inlet 533 of the blood collection container 530 or outlet 512 of the system housing 502 (or venturi outlet cone region 505c) can comprise a further system filter to further isolate and capture impurities mixed with blood transmitted through the gravity-driven filtering system 510.

The additional system filter can similarly comprise any of the aforementioned filter pore sizes, preferably, a pore size less than approximately 50.0 micron.

According to the invention, the inlet 533 of the blood collection container 530 or outlet 512 of the system housing 502 (or venturi outlet cone region 505c) can also comprise an emboli filter adapted to remove residual air, if any, from the processed autologous blood.

As indicated above, although the systems, apparatus and methods of the invention described above are primarily described in connection with processing autologous blood for reinfusion into a patient, the systems, apparatus and methods of the invention can also be readily employed to process non-autologous blood for transfusion into a patient.

In one aspect of the invention, there is thus provided a blood processing apparatus that is configured and adapted to process autologous and/or non-autologous blood for transfusion into a patient.

As indicated above, in some embodiments, the blood processing apparatus comprises a blood processing canister, the canister comprising an outer housing having an internal fluid passageway and a bottom blood collection chamber,
the internal fluid passageway sized and configured to receive blood therein and allow the blood to be transmitted therethrough, the internal fluid passageway in communication with the bottom blood collection chamber,
the internal fluid passageway comprising a venturi region, the venturi region comprising an inlet region, a restricted flow path region and an outlet region,
the venturi region further comprising a gravity-driven blood filtering system, the gravity-driven blood filtering system comprising a plurality of filters adapted to extract impurities from the blood, whereby purified whole blood is obtained, i.e., blood comprising all seminal native blood components; specifically, erythrocytes, platelets, white blood cells, plasma proteins, and antibodies, and substantially devoid of impurities,
the internal fluid passageway configured and adapted to transmit the purified whole blood to the bottom blood collection chamber.

As indicated above, in a preferred embodiment, blood flow through the internal fluid passageway and hence, venturi region exhibits a substantially laminar flow pattern, which minimizes the risk of shear forces imparted to blood and, hence, induced hemolysis of the erythrocytes in the blood.

As also indicated above, in a preferred embodiment, the purified whole blood comprises (i) an impurity content less than 0.05% and (ii) a retained content of all seminal native blood components, including native erythrocytes, platelets, white blood cells, plasma proteins and antibodies, of at least 98%.

As further indicated above, in a preferred embodiment, blood flow through the internal fluid passageway and, hence, gravity-driven blood filtering system comprises a flow rate of at least 500.0 cc/min.

In a preferred embodiment, the plurality of filters comprises a first filter, second filter, third filter and fourth filter.

In a preferred embodiment, the first filter comprises a pore size less than 10.0 mm, the second filter comprises a pore size less than 5.0 mm, the third filter comprises a pore size less than 300.0 micron, and the fourth filter comprises a pore size less than 100.0 micron.

In a preferred embodiment, the first filter, second filter, third filter and fourth filter comprise a conical shape comprising a cone angle in the range of approximately 25.0° to approximately 35.0° relative to a horizontal plane.

As also indicated above, an additional major advantage of the blood processing and reinfusion systems, apparatus and methods of the invention is that they can be promptly and readily employed during a multitude of surgical and interventional medical procedures, including, without limitation, invasive cardiac procedures, such as coronary artery bypass grafting (CABG), valve replacement and repair, and aortic aneurysm repair; orthopedic surgery procedures; spinal surgery procedures; neurosurgery procedures, such as craniotomy procedures; tumor resection procedures; organ transplant procedures; thrombectomy procedures; interventional cardiology procedures, such as percutaneous coronary intervention (PCI) and transcatheter aortic valve replacement (TAVR); interventional vascular procedures, such as endovascular aneurysm repair; interventional neurosurgery procedures, such as aneurysm coiling and arteriovenous malformation (AVM) procedures; and various trauma procedures.

Exemplar procedures using a blood reinfusion system of the invention are set forth below.

### Operating Room (OR) Procedures

### Stabilization of a Dysfunctional Sacroiliac (SI) Joint

A SI joint prosthesis, such as prosthesis 70 depicted and described in U.S. application Ser. No. 18/107,563, is provided.

The OR aspiration system is initially engaged to at least one suction canister, i.e., 200a, 200b (or both) of blood reinfusion system 100a, as depicted in FIG. 1.

An incision in and through tissue of the patient is made to provide posterior access to the patient's dysfunctional SI joint; preferably, a 2.0 cm to 3.0 cm incision.

The aspiration catheter, e.g., aspiration catheter 1000, is disposed proximate the incision site, i.e., body cavity formed via the incision, and engaged.

Thereafter, a guide bore is created in the dysfunctional SI joint, and a guide pin is inserted therein.

After the guide pin is inserted in the dysfunctional SI joint, a pilot opening is created in the dysfunctional SI joint with a tool assembly, the pilot opening comprising a first portion in the ilium bone structure and a second portion in the sacrum bone structure.

Thereafter, the tool assembly is removed, and the dysfunctional SI joint is flushed with a saline solution—the aspiration means continually aspirating the autologous blood of the patient, bone fragments, saline, etc. at the incision site and delivering the mixture of autologous blood and other impurities into and through the blood reinfusion system 100a for processing by the filter system 300.

After the tool assembly is removed and the SI joint is flushed, the prosthesis 70 is advanced into the pilot opening in the SI joint.

After the procedure is completed and before the incision is sutured, the aspiration catheter 1000 is removed from the incision site and the aspiration system is disengaged. The incision site is thereafter sutured and, hence, closed.

After the incision site is closed, the blood collection container 400a is disconnected from the filter system 300.

A transfusion line is thereafter attached to the outlet 409 of the blood collection container 400a and the blood collection container 400a is mounted on an IV stand.

Thereafter, the transfusion line is disposed in a blood vessel of the patient, wherein the processed autologous blood, i.e. purified whole autologous blood, is reinfused into the patient.

### Thrombectomy Procedures

As indicated above, the blood reinfusion systems, apparatus and methods of the invention can also be readily employed during thrombectomy procedures to remove occlusions and unwanted matter, such as thrombi or clots, from an artery or vein in a patient.

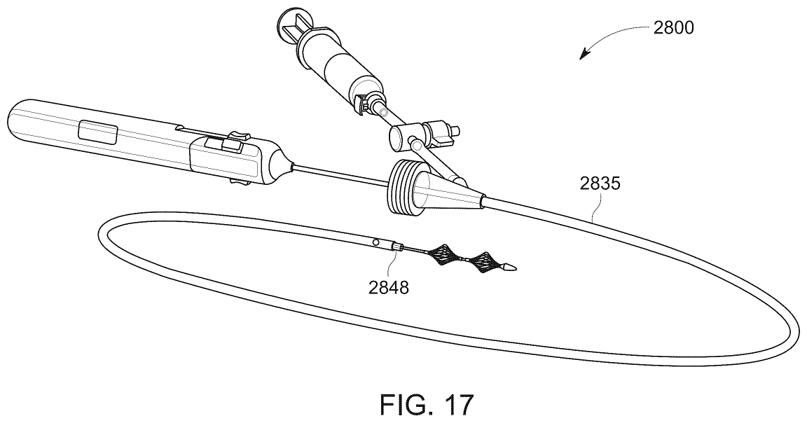

An exemplar thrombectomy procedure with the thrombectomy apparatus 2800 depicted in FIG. 17 (originally depicted in FIG. 28A of priority U.S. application Ser. No. 18/220,373 and referred to therein as a “retrieval apparatus”) is described below.

The OR aspiration system is initially connected to the aspiration catheter 2835 of the thrombectomy apparatus 2800. The aspiration catheter 2835 is thereafter connected to a blood reinfusion system of the invention, in this instance blood reinfusion system 100a.

The delivery catheter 2848 (with the aspiration catheter 2835 disposed therein, as described in priority U.S. application Ser. No. 18/220,373) is disposed in the patient's vessel, e.g., artery, proximate the occlusion, as described in priority U.S. application Ser. No. 18/220,373.

After the delivery catheter 2848 is disposed in the patient's vessel proximate the occlusion, the occlusion (and material thereof) is dislodged from the vessel with the thrombectomy apparatus 2800 and the occlusion (and material thereof) and autologous blood proximate thereto are aspirated into the aspiration catheter 2835, as described in U.S. application Ser. No. 18/220,373, and thereafter into the filter system 300, wherein the autologous blood is processed, in this instance, the occlusion (and material thereof) is filtered from the autologous blood.

After the occlusion (and material thereof) is dislodged from the vessel and aspirated into the aspiration catheter 2835, the delivery catheter 2848 is extracted out of the vessel.

After the delivery catheter 2848 is extracted out of the vessel, the blood collection container 400a is disconnected from filter system 300.

A transfusion line is thereafter attached to the outlet 409 of the blood collection container 400a and the blood collection container 400a is mounted on an IV stand.

Thereafter, the transfusion line is disposed in a blood vessel of the patient, wherein the processed autologous blood, i.e. purified whole autologous blood, is reinfused into the patient.

### EXAMPLES

The following examples are provided to enable those skilled in the art to more clearly understand and practice the present invention. The examples should not be considered as limiting the scope of the invention, but merely as being illustrated as representative thereof.

### Example I

### Evaluation of the Blood Reinfusion System's Ability to Filter Thrombosed Blood

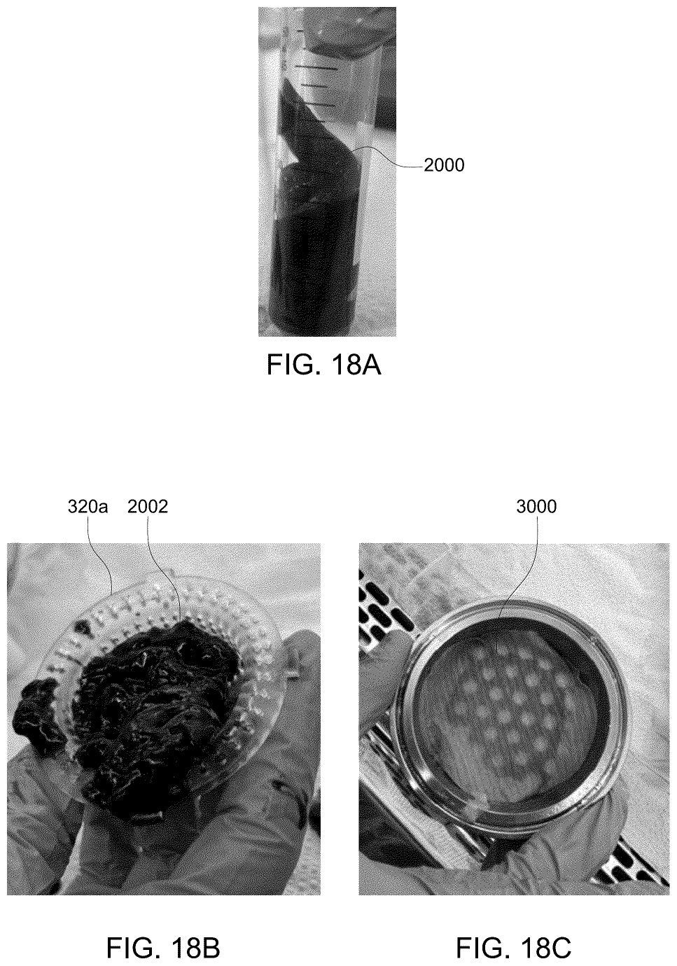

Referring now to FIG. 18A, thirty (30) cc of thrombosed bovine blood (denoted “2000”) was collected from the surgical site of a bovine animal. The thrombosed bovine blood was then combined with saline and drawn into a sixty (60) cc syringe. The 60 cc syringe containing the thrombosed blood and saline mixture was then connected to a blood delivery line in fluid communication with the blood reinfusion system 100a, depicted in FIGS. 1 and 2.

The thrombosed blood and saline mixture was then injected into and though the blood delivery line and introduced into the suction canister 200a of the blood reinfusion system 100a and into and though filters 320a, 320b, and 320c of the filter system 300.

Blood flow through the blood reinfusion system 100a comprised approximately 500.0 cc/min.

Another 60 cc syringe was then connected to the outlet 409 of the blood collection container 400a and the processed blood was drawn into the syringe.

A ten (10) cc portion of the blood processed with filter system 300 was then injected into a petri dish and visually examined for impurities to determine the filtration efficacy of the blood filter assembly 300 and, hence, blood reinfusion system 100a. The remaining portion of the processed blood was then filtered through a 40 μm filter (denoted 3000 in FIG. 18C) to confirm the absence of impurities.

Referring now to FIGS. 18B and 18C, there are shown the first filter 320a of the blood filter assembly 300 containing the impurities 2002 (FIG. 18B), in this instance thrombi, captured by the first filter 320a and the 40 μm filter 3000 (FIG. 18C) after the portion of the processed blood was filtered therethrough.

As depicted in FIG. 18C, the 40 μm filter 3000 was virtually devoid of impurities, and thus evidences the efficacy of filter system 300.

### Example II

### Evaluation of Erythrocyte Integrity and Morphology of Processed Porcine Blood

To evaluate the effect on erythrocyte integrity of porcine blood after processing with a reinfusion system of the invention, fifty (50) cc of untreated porcine blood was collected and divided into ten (10) cc and forty (40) cc samples. The 10 cc sample was left untreated and the 40 cc sample was processed via the blood reinfusion system 100a depicted in FIGS. 1 and 2 in accordance with the methods described herein.

The filtered 40 cc sample was then collected from the blood collection container 400a of the blood reinfusion system 100a for analysis.

The filtered 40 cc sample and the untreated 10 cc sample were then micro-histologically evaluated to determine erythrocyte integrity. Serum calcium (Ca), serum potassium (K) and hematocrit percent (Hct %) were determined for both the filtered 40 cc sample and the untreated 10 cc sample.

Micro-histologic evaluation of both the filtered 40 cc sample and the untreated 10 cc sample showed no significant differences in erythrocyte morphology. Based on the blood smear review, the erythrocytes and platelets in the filtered 40 cc sample and the untreated 10 cc sample similarly displayed no significant morphologic abnormalities.

As shown in Table I below, there also were no significant differences between the unfiltered control and the filtered sample in terms of serum potassium, serum calcium and Hct %.

The difference in platelet counts between the filtered 40 cc sample and the untreated 10 cc sample reflected in Table I were due to platelet clumping in the unfiltered sample.

TABLE I

| | SERUM K | SERUM Ca | HCT % | Platelets % |
|---|---|---|---|---|
| | mg/dl (Normal) | mg/dl (Normal) | (Normal) | (Normal) |
| CONTROL | 4.2 (3.5-5.5) | 10.0 (7.2-11) | 33 (28-40) | 152 (200 − |
| (UNFILTERED) | | | | 800 × 1000) |
| SAMPLE | 4.3 (3.5-5.5) | 10.1 (7.2-11.5) | 32 (28-40) | 181 (200 − |
| (FILTERED) | | | | 800 × 1000) |

### Example III

### Evaluation of Erythrocyte Integrity and Morphology of Processed Human Blood

To evaluate the effect on erythrocyte integrity of human blood after processing with a reinfusion system of the invention, fifty (50) cc of untreated human blood was collected and divided into ten (10) cc and forty (40) cc samples. The 10 cc sample was left untreated and the 40 cc sample was processed via the blood reinfusion system 100a depicted in FIGS. 1 and 2 in accordance with the methods described herein.

The filtered 40 cc sample was then collected from the blood collection container 400a of the blood reinfusion system 100a for analysis.

The filtered 40 cc sample and the untreated 10 cc sample were then micro-histologically evaluated to determine erythrocyte integrity. Lactate dehydrogenase (LDH), total bilirubin, aspartate aminotransferase (AST), alanine transaminase (ALT), albumin, serum potassium (K), hematocrit percent (Hct %) and platelet concentration were similarly determined for the filtered 40 cc sample and the untreated 10 cc sample.

Micro-histologic evaluation of both the filtered 40 cc sample and the untreated 10 cc sample similarly showed no significant differences in erythrocyte morphology. The erythrocytes and platelets in the filtered 40 cc sample and the untreated 10 cc sample similarly reflected no significant morphologic abnormalities.

As shown in Table II below, there were also no significant differences between the unfiltered control and the filtered sample in terms of total bilirubin, aspartate aminotransferase (AST), alanine transaminase (ALT), albumin, serum potassium (K), hematocrit percent (Hct %) and platelet concentration.

TABLE II

| | CONTROL UNFILTERED | | SAMPLE FILTERED | |
|---|---|---|---|---|
| | (Normal) | | (Normal) | |
| LDH | 171 U/L | (120-246) | 323 U/L | (120-246) |
| TOTAL | 0.5 mg/dL | (0.2-1.1) | 0.4 mg/dL | (0.2-1.1) |
| BILIRUBIN | | | | |
| AST | 27 U/L | (0-34) | 32 U/L | (0-34) |
| ALT | 43 U/L | (10-49) | 44 U/L | (10-49) |
| ALBUMIN | 4.9 g/dl | (3.2-4.8) | 4.6 | (3.2-4.8) |
| K | 4.7 mmol/L | (3.5-5.1) | 4.6 mmol/L | (3.5-5.1) |
| HCT | 49.4% | (40-51) | 47% | (40-51) |
| PLATELET | 286 K/μL | (150-400) | 285 K/μL | (150-400) |

| | | |
|---|---|---|
| BLOOD SMEAR | Normal | Normal |

### Example IV

### Evaluation of Gravity-Driven Filtration Efficacy of Filter System

In order to assess the gravity-driven filtration efficacy of a filter system of the invention, in this instance filter system 300, a transparent, high-viscosity (˜0.003-0.006 pascal-seconds (Pa*s)) whole blood analog solution was prepared that comprised 50.0 cc of distilled water, 50.0 cc of glycerol, 2.3 mm stainless steel spherical particles, 355.0-425.0 μm red, fluorescent spherical particles, and 63.0-75.0 μm green, fluorescent spherical particles.

The whole blood analog solution was then injected into and though the blood delivery line and introduced into the suction canister 200a of the blood reinfusion system 100a and into and though filters 320a, 320b, and 320c and into the second reservoir 316 of the filter system 300.

The flow of the whole blood analog solution into and through the blood reinfusion system 100a was >500.0 cc/min.

The filter system 300 was then disassembled and the filters 320a, 320b, and 320c were then extracted from the system 300 and inspected under ultraviolet light. Filter 320a contained virtually all of the 2.3 mm stainless steel spherical particles, filter 320b contained virtually all of the 355.0-425.0 μm red, fluorescent spherical particles (and a portion of the 63.0-75.0 μm green, fluorescent spherical particles) and filter 320c contained virtually all of the 63.0-75.0 μm green, fluorescent spherical particles.

The filtered whole blood analog solution collected in the second reservoir 316 of the filter system 300 was also inspected under ultraviolet light and did not contain any visible spherical particles.

Inspection of the filtered whole blood analog solution and filters 320a, 320b, and 320c of the filter system 300 thus confirmed the gravity-driven filtration efficacy of the system 300.

Thus, as will readily be appreciated by one having ordinary skill in the art, the present invention provides numerous significant advantages compared to prior art blood reinfusion systems and methods. Among the advantages are the following:
the provision of improved blood reinfusion systems, apparatus and methods adapted to process autologous blood with minimal, if any, effect on the quality of the blood;
the provision of improved blood reinfusion systems, apparatus and methods adapted to process autologous blood with minimal, if any, effect on total bilirubin, aspartate aminotransferase (AST), alanine transaminase (ALT), albumin, serum potassium (K), hematocrit percent (Hct %) and platelet concentration;
the provision of improved blood reinfusion systems, apparatus and methods adapted to process autologous blood with minimal blood component loss; specifically, erythrocyte, platelet, white blood cell, plasma protein and antibody loss;
the provision of improved blood reinfusion systems, apparatus and methods that can be employed in sterile environments;
the provision of improved blood reinfusion systems and apparatus that are simple to use and can be easily operated manually by a single operator;
the provision of improved blood reinfusion systems and apparatus that can be promptly employed in emergency situations;
the provision of improved blood reinfusion systems, apparatus and methods that can be employed in a multitude of surgical and interventional medical procedures; and
the provision of blood processing apparatus adapted to process blood for transfusion into a patient.

Without departing from the spirit and scope of this invention, one of ordinary skill in the art can make various changes and modifications to the invention to adapt it to various usages and conditions. As such, these changes and modifications are properly, equitably, and intended to be, within the full range of equivalence of the following claims.

What is claimed is:

1. A blood reinfusion system, comprising:
a blood processing canister comprising a housing, said housing comprising an internal fluid passageway and a bottom blood collection chamber,
said internal fluid passageway sized and configured to receive blood therein and allow said blood to be transmitted therethrough, said internal fluid passageway in communication with said bottom collection chamber,
said internal fluid passageway comprising a venturi region, said venturi region comprising an inlet region, a restricted flow path region and an outlet region, said inlet region of said venturi region defining a top blood collection chamber,
said venturi region comprising a first length,
said inlet region comprising a cone angle in the range of 26° to 66° and a second length in the range of 25% to 35% of said first length of said venturi region,
said outlet region comprising a cone angle in the range of 10° to 45° and a third length in the range of 75% to 85% of said first length of said venturi region,
said venturi region further comprising a gravity-driven blood filtering system adapted to extract a plurality of impurities from said blood, whereby purified whole blood is obtained,
said internal fluid passageway configured and adapted to transmit said purified whole blood to said bottom blood collection chamber.

2. The system of claim 1, wherein flow of said blood through said venturi region exhibits a laminar flow pattern, whereby hemolysis of erythrocytes in said blood is abated.

3. The system of claim 1, wherein said purified whole blood purified whole blood comprises native erythrocytes, platelets, white blood cells, plasma proteins and antibodies.

4. The system of claim 1, wherein retained content of said native erythrocytes, platelets, white blood cells, plasma proteins and antibodies in said purified whole blood is at least 98%.

5. The system of claim 1, wherein impurity content of said plurality of impurities in said purified whole blood is less than 0.05%.

6. The system of claim 1, wherein said gravity-driven blood filtering system comprises a plurality of filters.

7. The system of claim 6, wherein said plurality of filters comprises a first filter adapted to extract first impurities of said plurality of impurities from said blood.

8. The system of claim 7, wherein said first filter comprises a pore size less than 10.0 mm.

9. The system of claim 7, wherein said plurality of filters comprises a second filter in communication with said first filter, said second filter adapted to receive said blood from said first filter and extract second impurities of said plurality of impurities from said blood.

10. The system of claim 9, wherein said second filter comprises a pore size less than 5.0 mm.

11. The system of claim 9, wherein said plurality of filters comprises a third filter in communication with said second filter, said third filter adapted to receive said blood from said second filter and extract third impurities of said plurality of impurities from said blood.

12. The system of claim 11, wherein said third filter comprises a pore size less than 300.0 micron.

13. The system of claim 11, wherein said plurality of filters comprises a fourth filter in communication with said third filter, said fourth filter adapted to receive said blood from said third filter and extract fourth impurities of said plurality of impurities from said blood.

14. The system of claim 13, wherein said fourth filter comprises a pore size less than 100.0 micron.

15. The system of claim 13, wherein said first filter comprises a first top surface area, said second filter comprises a second top surface area, said third filter comprises a third top surface area and said fourth filter comprises a fourth top surface area.

16. The system of claim 13, wherein said second filter, said third filter and said fourth filter are disposed in said outlet region of said venturi region, whereby said fourth top surface area of said fourth filter is greater than said third top surface area of said third filter, and said third top surface area of said third filter is greater than said second top surface area of said second filter.

17. The system of claim 16, wherein said second filter, said third filter and said fourth filter comprise a conical shape.

18. The system of claim 1, wherein said bottom blood collection chamber is detachably coupled to said blood processing canister.
